(19) United States
(12) Patent Application Publication
(10) Pub. No.: US 2023/0131234 A1
(43) Pub. Date: Apr. 27, 2023

(54) SYSTEMS AND METHODS FOR PREDICTING OUTCOMES AND CONDITIONS OF CHEMICAL REACTIONS WITH HIGH RELIABILITY BASED ON A HIGHLY DIVERSE AND ACCURATE DATASET

(71) Applicant: Molecule One Sp. z o.o., Warsaw (PL)

(72) Inventors: Stanislaw Jastrzebski, Warsaw (PL); Mateusz Bruno-Kaminski, Krakow (PL); Jan Busz, Warsaw (PL); Piotr Byrski, Warsaw (PL); Artur Choluj, Warsaw (PL); Pawel Dabrowski-Tumanski, Warsaw (PL); Tomasz Dybowski, Warsaw (PL); Piotr Helm, Warsaw (PL); Marek Pietrzak, Namyslow (PL); Szymon Pilkowski, Warsaw (PL); Jan Rzymkowski, Warsaw (PL); Michal Sadowski, Warsaw (PL); Lukasz Szczupak, Lodz (PL); Mikolaj Sacha, Krakow (PL); Filip Ulatowski, Malcanow (PL); Ruard van Workum, Arnhem (NL); Paulina Wach, Warsaw (PL); Przemyslaw Pobrotyn, Warsaw (PL); Pawel Wlodarczyk-Pruszynski, Warsaw (PL)

(73) Assignee: Molecule One Sp. z o.o., Warsaw (PL)

(21) Appl. No.: 18/048,981

(22) Filed: Oct. 24, 2022

Related U.S. Application Data
(60) Provisional application No. 63/270,932, filed on Oct. 22, 2021.
(60) Provisional application No. 63/351,295, filed on Jun. 10, 2022.

(51) Int. Cl. G16C 20/10; G16C 20/50; G16C 20/20

(52) U.S. Cl. CPC G16C 20/10; G16C 20/50; G16C 20/20

(57) ABSTRACT

Methods and systems are disclosed in which an automated or semi-automated laboratory may be combined with a machine learning methodology to enable predicting outcomes of chemical reactions or to predict reaction conditions. The model may be trained on reactions including data from the laboratory, purposefully selected to satisfy a desired goal by a user. The user can interact with the process and the model via dedicated user interfaces designed to enable efficient user-machine interaction. The method can be used in the context of multiple challenging problems in chemistry such as steering an automated chemistry laboratory, synthesizing a large collection of compounds such as DNA encoded library, or recommending high yielding reaction conditions for reactions involving drug-like compounds.

### CROSS-REFERENCE TO RELATED CASES

This application claims priority to U.S. Provisional Patent Application No. 63/270,932, entitled “Trust-Worthy Systems And Methods For Discovering Novel Chemical Reactions Or Classes Of Reactions, Or Optimization Of Existing Reactions,” filed on Oct. 22, 2021; and U.S. Provisional Patent Application No. 63/351,295, entitled “Tool For Recommending Chemical Reaction Conditions,” filed on Jun. 10, 2022, both of which are hereby incorporated by reference. This application is related to U.S. patent application Ser. No. 17/060,765, entitled “Systems And Method For Designing Organic Synthesis Pathways For Desired Organic Molecules,” filed on Jan. 14, 2021.

### BACKGROUND

Predicting outcomes of chemical reactions is a central task to a plethora of industries that use chemistry such as drug discovery, agriculture or cosmetics. Consider drug discovery. For every drug in the market, usually thousands have to be synthesized and tested in the laboratory. Any inefficiency in the chemical processes used—for example failing to obtain desired products in the laboratory—can have a significant downstream effect, increasing the prices of goods and services.

Unfortunately, predicting outcomes of many classes of chemical reactions is hard for humans and computers alike. For example, a 55% internal failure rate of Buchwald-Hartwig coupling reactions has been reported based on internal industry data. The task is hard for computers as well. Despite significant progress in computational chemistry, it will be some time before simulations can replace experiments. It may be possible to accurately predict outcomes of specific chemical reactions from first principles when (a) the reaction mechanism is well understood; (b) the reaction mechanism is simple (for example includes only one transition state), and (c) the overall chemical complexity of the involved reactants and reagents is low.

Another approach to predicting reaction outcomes is based on machine learning, and deep neural networks (DNNs) in particular. Machine learning methods however are fundamentally limited by the available data. Deep neural networks trained on publicly available data generalize poorly due to inherent biases in publicly available data in particular, almost all sources of data completely omit failed experiments.

However, current approaches are limited in terms of accuracy predicting outcomes of reactions for a broad set of chemical space. One of the limiting factors is the fact that most of the previous work has considered a very low coverage of the molecular space. For example, Shields et al mostly consider reaction spaces with only a few or only a single reaction product.

The inability to predict reaction outcomes results, in particular, in increased costs and lengthened timelines for introducing new drugs into the market. Thus, what is needed are systems and methods for predicting outcomes and conditions of chemical reactions with high accuracy and reliability.

### BRIEF DESCRIPTION OF THE DRAWINGS

The embodiments are illustrated by way of example and not limitation in the figures of the accompanying drawings, in which like references indicate similar elements, and in which:

FIG. 1 is a screenshot of an embodiment of a graphical user interface (GUI) to an embodiment of a model for predicting outcomes and conditions of chemical reactions;

FIG. 2 is a screenshot of the embodiment of a GUI to an embodiment of a model for predicting outcomes and conditions of chemical reactions showing the creation of a new functionalization;

FIG. 3 is a screenshot of the embodiment of a GUI to an embodiment of a model for predicting outcomes and conditions of chemical reactions showing a functionalization search exploration overview;

FIG. 4 is a screenshot of the embodiment of a GUI to an embodiment of a model for predicting outcomes and conditions of chemical reactions showing an exploration overview marked location hover state;

FIG. 5 is a screenshot of the embodiment of a GUI to an embodiment of a model for predicting outcomes and conditions of chemical reactions showing an exploration overview results filtered by functionalization type;

FIG. 6 is a screenshot of the embodiment of a GUI to an embodiment of a model for predicting outcomes and conditions of chemical reactions showing a functionalization detail view;

FIG. 7 is a screenshot of the embodiment of a GUI to an embodiment of a model for predicting outcomes and conditions of chemical reactions showing a functionalization detail expanded reference reaction;

FIG. 8A and FIG. 8B are first and second halves, respectively, of a flowchart illustrating an embodiment of method for predicting outcomes and conditions of chemical reactions;

FIG. 9 is a screenshot of the embodiment of a GUI to an embodiment of a model for predicting outcomes and conditions of chemical reactions;

FIG. 10 is a screenshot of the embodiment of a GUI to an embodiment of a model for predicting outcomes and conditions of chemical reactions;

FIG. 11 is a screenshot of the embodiment of a GUI to an embodiment of a model for predicting outcomes and conditions of chemical reactions;

FIG. 12 is a diagram illustrating forms of input and output in an embodiment of a GUI to an embodiment of a model for predicting outcomes and conditions of chemical reactions;

FIG. 13 is a flowchart illustrating an embodiment of a data collection method for a model for predicting outcomes and conditions of chemical reactions;

FIG. 14 is a screenshot of an advanced query builder in an embodiment of a GUI to an embodiment of a model for predicting outcomes and conditions of chemical reactions;

FIG. 15 is a screenshot of the depiction of reference reactions in an embodiment of a GUI to an embodiment of a model for predicting outcomes and conditions of chemical reactions;

FIG. 16 is a screenshot of a reaction editor in an embodiment of a GUI to an embodiment of a model for predicting outcomes and conditions of chemical reactions;

FIG. 17 is a flowchart illustrating an embodiment of a method for predicting outcomes and conditions of chemical reactions;

FIG. 18 is an exemplary block diagram depicting an embodiment of a system for implementing embodiments of methods of the disclosure; and

FIG. 19 is an exemplary block diagram depicting a computing device.

### DETAILED DESCRIPTION

A goal of the disclosed subject matter is to obtain machine learning models that can accurately predict outcomes of reactions on broad and commercially valuable compounds. The innovation is designed to enable addressing hard problems in chemistry such as recommending high yielding conditions for reactions such as Suzuki coupling or Heck coupling.

There are several embodiments geared towards achieving high accuracy on broad and commercially valuable compounds. Notably, a semi-automated high-throughput laboratory is used, which enables generating large datasets of chemical reactions. Another innovation is prioritizing reactions (for execution in the laboratory) using novel methods focused on achieving high accuracy on user-relevant reactions.

In some embodiments, a cost-effective and high-throughput (HT) organic chemistry laboratories may be used in a method for predicting outcomes and conditions of chemical reactions with high accuracy and reliability that is based on creating a large focused dataset of chemical reactions for training a machine learning model. Such embodiments may include a process by which a model (computer program) learns to accurately and reliably predict outcomes of reactions on broad and commercially valuable compounds with high accuracy and a good estimation of uncertainty. Such a model may be applied to predict difficult problems in, e.g., organic chemistry.

Embodiments may employ a high-throughput laboratory designed with two key constraints, (a) low cost per reaction (e.g., <1$ per reaction), and (b) high throughput (e.g., 5000 reactions per week). For example, the cost constraint may be addressed by sourcing building blocks from large scale providers, such as MolPort.

In one embodiment, to ensure that the model can generalize to a broad range of drug-like molecules, experimental reactions are chosen such that they include reactions particularly relevant for a target set that includes drug-like molecules, as described later in this document. Choosing such a focused set of experimental reactions may significantly boost performance with respect to drug-like molecules because drug-like molecules have highly biased structures, which may be covered by a relatively small number of focused experiments. In some embodiments, the target set may be specified to consist of any type of molecules most relevant for a given application. In some embodiments, to better train the model so that it can generalize to a broad range of molecules within a particular chemical space, experimental reactions within that chemical space are chosen that represent the breadth of the chemical space for the target set.

In some embodiments, methods are disclosed for training machine learning models to be able to produce results that are robust enough to directly inform experimental design within particular reaction classes.

Some embodiments include the purposeful creation of datasets of chemical reactions that are then used to train models that are highly specialized in particular chemical reaction classes. In addition, embodiments may include, the use of medium- or high-throughput chemistry experiments to do the above, the use of DNA-encoded libraries to do the above; the use of MALDI-TOF mass spectrometry to do the above; the use of MISER chromatography to do the above; the use of an automated chemistry laboratory to do the above, the use of proprietary data mining algorithms to do the above.

Some embodiments may include an intuitive graphical user interface that enables: querying the system about desired chemical reactions, intuitively viewing system's recommendations, consulting extensive supporting information that give the user unprecedented confidence in the results, which may include among others. ML model outputs, and proprietary experimental datasets (mentioned above).

Some embodiments may combine all of the above in a methodology/computer system that enables user-machine and machine-machine interaction with the goal of: identifying ways to execute certain chemical reactions, including ways that are better in some regard, e.g. have higher yield; or identifying chemical reactions that are executable with certain user-defined constraints, such as limited range of conditions; or identifying chemical reactions that can be directly sent to an automated synthesis system for execution.

In some contexts, “machine-machine interaction” means outputting the results of the system directly into another computer system, such as one governing an automated laboratory.

Some embodiments may include the use of one or more of the aforementioned methodologies in the fields of: late-stage functionalization of compounds in the drug discovery pipeline (such an embodiment is described in more detail later in this disclosure) and predicting the conditions of chemical reactions (described in more detail later in this disclosure).

Some embodiments may include the use of one or more of the aforementioned methodologies in the field of DNA-encoded library (DEL) synthesis. Such an embodiment includes applying the strategy in steps 1-3 as described in more detail below to identify reaction conditions that enable creating more diverse or more efficient DELs. This may help in excluding certain chemical reactions or certain conditions of chemical reactions that are generally known, but that are not acceptable in the context of DEL synthesis. In such an embodiment, the methodology of Steps 1-3 (discussed below) may be used to improve the efficiency of DEL synthesis through better selection of substrates that will successfully undergo a certain reaction In the context of DEL synthesis this is important because of the large scale and high purity standards that are necessary. In another such embodiment, a given chemical reaction may be optimized for applicability in conditions suitable for DEL synthesis by application of the methodology in steps 1-3. DEL synthesis benefits from performing chemical reactions in mild conditions that do not negatively affect the DNA tags (e.g. by causing the DNA to disintegrate).

Some embodiments may include the use of one or more of the aforementioned methodologies in the field of automating execution of chemical reactions with the use of robots. Whereas typical approaches may focus on hardware or specific applications of automated synthesis, the approach of embodiments presented in this disclosure focuses on the comprehensive coverage of a part of the chemical space with the use of large reaction databases, and/or developing models that use these databases to make accurate predictions. This is distinctive because it addresses the key problem that other procedures for automatically executing chemical reactions have, which is that the user that has to program the robots by setting substrates, conditions, and other parameters necessary for execution. In one such embodiment, the method of steps 1-3 focuses on establishing conditions for performing certain chemical reactions that maximize scope, i.e. under the same conditions a given reaction produces satisfying yield for a large number of different substrates.

In embodiments a method enables predicting novel chemistry in a way that is trust-worthy for chemists. In such contexts, “novel chemistry” means one or more of the following: newly discovered classes of chemical reactions; expanding the molecular scope of known reactions classes; enabling the synthesis of novel compounds; increasing the yield of a known reaction: or discovering new conditions for a known chemical reaction.

In some embodiments, a method is comprised of the following Steps 1-3.

Step 1) creating a detailed dataset of chemical reactions that focuses on one or more classes of chemical reactions that are of interest to the user of the methodology, and is broad enough to enable predicting outcomes of selected novel chemical reactions with large confidence. Such a dataset may be created based on a combination of one or more of the following methodologies (a . . . g).

a. Automatically or manually parsing text data about reactions (e.g. literature, textbooks, patents, laboratory notebooks, or Internet websites) in order to extract chemical reactions (which may be a part of the dataset). In such parsing, i) generally known techniques may be modified to execute the chemistry of interest. Certain novel elements are included in the embodiment description. In such parsing, ii) the dataset may be enriched using manually specified reaction rules that can be used to automatically generate artificial reactions. Such rules may include information such as the type of transformation, its scope, required reaction conditions. In such parsing, iii) external datasets of molecules or external datasets of chemical reactions may also be used to enrich the dataset. In particular, chemical reactions extracted from patents filed with the United States Patent and Trademark Office may be used for that purpose.

b. Using medium- and high-throughput analytics techniques such as MALDI-TOF, and MISER. MALDI-TOF is a generally known technique of mass spectrometry, which is in turn a range of techniques used to identify chemical compounds. One feature of MALDI-TOF that is key for the purpose of this methodology is its capability of achieving high throughputs (below is per sample). In embodiments, this technique may require adaptation to create datasets of interest. Applying this technique in creating datasets of interest benefits from using proper analytical procedure including, but not limited to the choice of matrix composition and ionization setup. MISER chromatography coupled with MS detection is a generally known technique of compound separation and identification. One feature that is helpful for the purpose of this methodology is its capability of achieving medium-to-high throughputs (below 30s per sample) This technique may also require adaptation to create datasets of interest. In embodiments, these techniques may be used to create a dataset of reaction outcomes that is of sufficient size and quality to power a system such as the one described in this disclosure. In particular, this may be facilitated by automatically feeding these datasets to machine learning models that are predicting the outcomes of other chemical reactions. In embodiments, deep neural networks may be used to denoise or increase fidelity of analytical results produced by high-throughput analytics techniques such as MALDI-TOF or MISER. In particular, a neural network may be trained to predict a full mass spectrogram of a molecule based on the output from MALDI-TOF or MISER. For example, in an embodiment, machine learning methods may be used to predict the ionizability of compounds analyzed with MALDI-TOF or other MS methods, based on data about the ionizabiiity of other compounds with known ionizabiiity, thus improving the accuracy (“quantitativeness”) of these analytical methods.

c. Using medium- and high-throughput chemistry experiments. Some generally known techniques may be modified to execute the reactions of interest. These techniques may be used to create a dataset of sufficient size and quality to power a system such as the one described in this disclosure. Furthermore, in embodiments, the user is enabled to execute chemical reactions themselves to verify outcomes of the system. Furthermore, in some embodiments, the experiments may be performed with the aid of automated liquid and/or solid dispensers.

d. Using DNA-encoded libraries. In some embodiments, a DNA-encoded library (DEL) may be used as means for generating experimental data on reactivity of DNA-tagged reagents, which may be relevant for training machine learning models (and the Model in particular). In one embodiment of this kind, a library of reagents bearing a common functional group, each tagged with a different DNA tag is used. A mixture of such tagged library components (A) is allowed to undergo a chemical reaction with (a) certain reagent(s) (B), which results in formation of covalent bonds between some elements of A and some elements of B. Proper construction of reagent B (tagging or immobilization) enables subsequent cheap identification of formed A-B adducts. In an embodiment, the reagent B may be attached to a large molecule such a DNA strand, protein (polypeptide), nano-particle, or polymeric resin bead, which enables washing out the unreacted library components A and subsequent identification of DNA tags of the components of library A that underwent the reaction with reagent B, using widely known techniques, such as polymerase chain reaction (PCR) and next generation sequencing (NGS).

e. Using simulation software. In an embodiment, molecular simulation software may be used to predict outcomes of chemical reactions in order to enrich the dataset. In an embodiment, simulation software may be used to predict outcomes of chemical reactions for simple molecules, for which it provides sufficiently accurate results, to bootstrap learning of machine learning models that predict outcomes of more complex reactions.

f. Experimentation with existing literature data and existing machine learning models to discover what experiments are most informative in the context of the chemistry of interest, and testing whether the created dataset is already detailed enough. For example, in an embodiment, i., machine learning models may be used to discover the most useful reactions to perform to enable more trustworthy predictions. In particular, machine learning certainty estimates may be used to select the most uncertain reactions. Reactions for which the model is most uncertain about its predictions can be performed and analyzed in a lab in order to add them to the training set and thus enhance the mode). Additionally, in embodiments, ii., existing literature data or other sources of information may be used to determine whether the dataset is already detailed enough for the chemical goal of interest. In one such embodiment, existing sources of information may be reviewed in terms of how many data points they contain on the chemistry of interest. Such an analysis can be performed by a chemist, a statistical model or a combination of both. In another such embodiment, these sources may be used as training datasets for machine learning models, such as the one referenced below in step 2a. Additionally, in embodiments, iii., the space of chemical reactions may be divided into groups using broad chemical features (calculated using generally known chemical software and machine learning models). For example, in an embodiment, from these groups of chemical reactions, a smaller number of groups may be selected such that, when performed and analyzed in a lab, these selected groups would give the most useful training data for the machine learning algorithms in order to enhance their robustness in predicting the outcomes of a pool of chemical reactions coming from other groups. In another example, in an embodiment, groups of reactions may be selected for which the outcomes of such reactions are relatively the hardest to predict without the use of a robust machine learning system. Selected groups may then be used to design the experiment in such a way that from each group of interest, chemical reactions are densely sampled. Such experimental design enables training more robust machine learning models. In another embodiment, iv., synthesis planning software may also be used to investigate which reactions would enable reaching given molecules of interest.

g. Manual labeling of reactions generated by machine learning software by chemists. In an embodiment, i., reaction candidates may be generated by a computer system using methods described below in “Late stage functionalization,” subsection 3a, and then given to one or more chemists that are instructed to assign labels for reactions.

In embodiments, the various techniques described above (1.a . . . 1.g.) may be combined in the context of creating a dataset of chemical reactions that may then be used to train a machine learning model focused ort identifying trust-worthy chemistry (Step 2) and continuously updating the dataset with new reaction data that is carefully selected to maximize its robustness with minimal costs of laboratory experiments.

Step 2) training a machine learning model on the created detailed dataset, and any other relevant sources of information, that is focused on identifying trust-worthy chemistry.

a. In embodiments, the machine learning model may be any model, trained with use though not exclusive use, of the created dataset in Step 1, that is able to predict the chemistry of interest. In particular, if the chemistry of interest requires predicting detailed conditions under which to perform reaction (such as reagents, solvent, temperature), it is included in the output of the model.

b. In embodiments, a machine learning model is created that is focused on making trust-worthy predictions for novel (unseen) chemistry (e.g. novel molecules). The system is designed to show predictions that are highly confident, at the expense of the number of predictions shown.

c. In embodiments, the machine learning model may also be trained in such a way that it is able to make sufficiently confident predictions, which may be provide by using one or more of: i., the detailed dataset created in Step 1, ii. training on additional datasets of molecules or reactions so that the model is exposed to a broader knowledge about what molecules exist; and iii. ensembling or other techniques used to increase inter-domain generalization (causal learning in one embodiment).

d. In embodiments, these techniques may be used to train a machine learning model that makes confident predictions about reaction outcomes (Step 2).

Step 3) finding how to perform a chemical reaction of interest by combining one or both of: a or b a. A user-friendly interface is adapted to the specific chemistry of interest and focused on enabling efficient user-machine interaction, for example, i., a model that is trust-worthy may be combined with a detailed experimental dataset that a chemist can trust in. ii. The user is enabled to discuss and compare the results to literature and the created dataset (in Step 1) by being able to explore the most chemically related chemical reactions from both sources. In an embodiment of ii, these related chemical reactions are chosen through fingerprint similarity of the product of the reaction of interest to the products of potentially related reactions. In another embodiment of ii, these related chemical reactions are chosen based on similarity of molecular features of the product of the reaction of interest to the products of potentially related reactions. Examples of such molecular features include: the presence of functional groups, the order of atom where the reaction is happening, and intra- or intermolecular character of the reaction

b. Automated synthesis platform. In embodiments, the user is enabled to suggest and execute chemical reactions that experimentally verify novel predictions.

In embodiments, the procedure of steps 1-3 may be applied iteratively, with Step 1 using outcomes resulting from Step 3, to improve the outcomes of each step.

### Late Stage Functionalization

In an embodiment of the proposed methodology, robust machine learning models, a dataset based on literature (which is sourced using a custom multi-stage extraction pipeline), and an intuitive GUI are combined and applied to late stage functionalization. Late stage functionalization is a methodology in the drug discovery process, where a promising drug candidate is optimized by making small modifications to its structure. Every new structure is immensely valuable, as it benefits from the activity data available for its close analogue. However, performing late stage functionalization comes with a substantial challenge of finding reaction conditions that allow to perform the transformation chemoselectively.

Embodiments provide the user with access to highly trust-worthy predictions on how the molecule can be modified and under what conditions, in turn giving them access to a larger range of analogues than available without using the embodiment.

In one such embodiment, 1. the machine learning model is a neural network based on the Transformer architecture (in other embodiments the model may be based on other model architectures as well), and is trained using some of the techniques described above in Step 2, to enable trust-worthy predictions for the chemistry of interest.

2. In another such embodiment, the model may be trained using: a. a technique called self-supervised learning using data extracted from publicly available documents (e.g., patents) that contain information about chemical reactions that were successfully performed in the past. The details of these chemical reactions may be extracted using machine learning methods that create an extensive, detailed and robust dataset, i. The extraction of chemical data is performed using a pipeline of machine learning models that parse data in several stages. 1. A first stage using a model that predicts whether a fragment of text describes a chemical reaction. 2. A second stage using a model that labels within a chemical reaction description paragraphs as headers, descriptions or footers 3. A third stage using a model that predicts which parts of a reaction description are names of entities such as. reaction product, substrates, solvents, catalysts and other conditions necessary to perform the reaction, ii. In an embodiment, all stages of the reaction extraction pipeline may be performed by neural networks based on the Transformer architecture trained specifically for each task using manually labeled datasets. These are other types of models than those used for chemistry-related tasks (such as the one mentioned in embodiment 1. above at the beginning of this paragraph). The models mentioned in embodiment 2, section i. 1-3 are trained specifically for natural language processing tasks iii The detailed reaction properties predicted by the pipeline improve the efficacy of using the aforementioned self-supervised training technique. The model may be trained using b. a supervised learning objective based on the same data as in the previous point, or c a large number of automatically generated artificial chemical reactions that are probably incorrect (i.e. probably would not work in the laboratory).

3. For embodiments predicting novel chemistry, only highly confident predictions are selected from the model output. To achieve this, the model predicts as a separate output its confidence, and only chemical reactions for which the value of this output is above a predefined threshold are selected. For example, chemical reactions may be generated either by a generative model (a model that generates substrates based on the product, or the product based on substrates) or using so-called reaction templates. In the latter case, the final confidence level may be calculated using a discriminator model (a classification model that outputs a single value indicating probability of a chemical reaction succeeding) that was trained on examples of positive and negative examples.

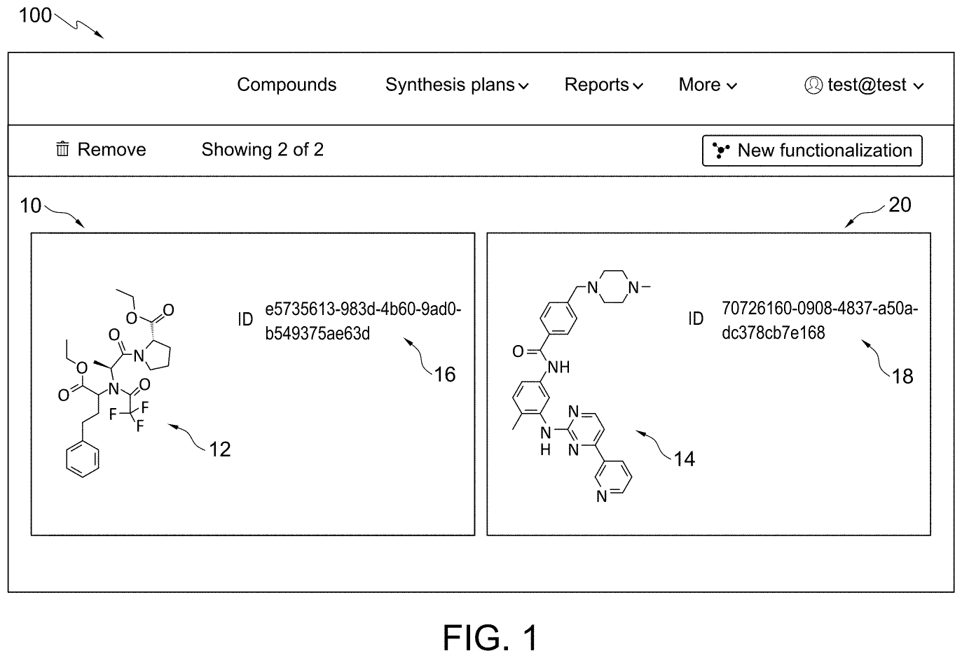

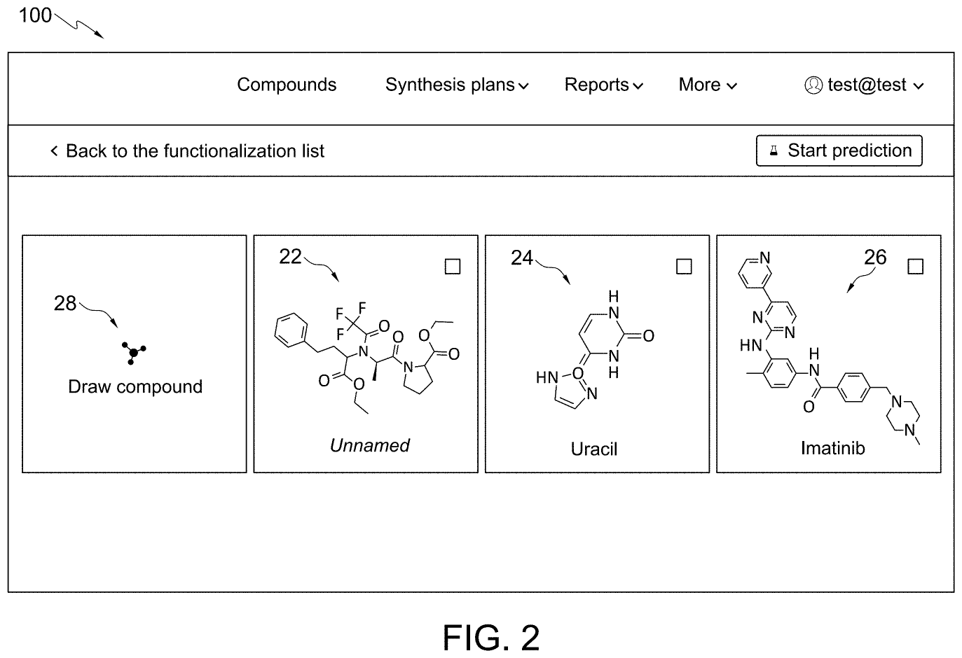

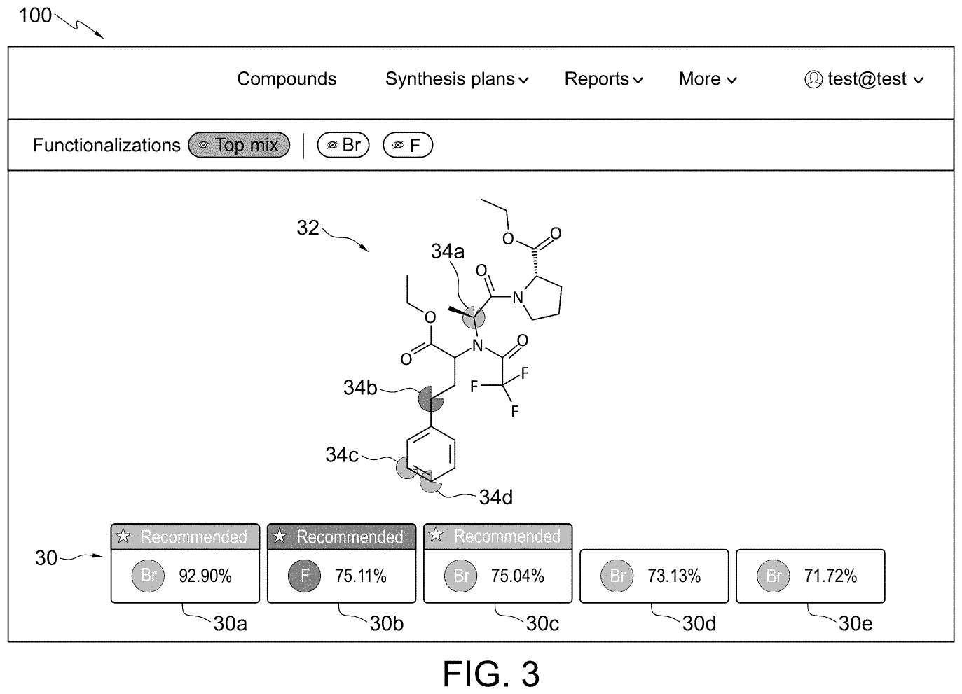

FIG. 1 is a screenshot of an embodiment of a GUI to an embodiment of model for predicting outcomes and conditions of chemical reactions. In FIG. 1, searches 10, 20 are each a functionalization search previously performed in the system Molecules 12, 14 illustrate molecules on which the search was previously performed. And a short summary 16, 18 is provided for each search. In the GUI, selecting an item in one of searches 10, 20 redirects to the search exploration page (FIG. 3) for the related item. A “New Functionalization” button provides the ability to create a new functionalization (FIG. 2). In this screen, like atoms may be colored similarly, e.g., F atoms in molecule 12 may be light blue and “O” atoms may be red.

FIG. 2 is a screenshot of the embodiment of a GUI to an embodiment of model for predicting outcomes and conditions of chemical reactions showing the creation of a new functionalization. Molecules 22 (previously identified as molecule 12), 24, 26 illustrate molecules that may be selected to perform the search on. A new molecule may be created by selecting new compound 28. After the compound is selected the search (prediction) can be started by selecting “start prediction.” In this screen, like atoms and like function groups may be colored similarly, e.g., F atoms in molecule 22 may be colored light blue and the “NH” group in molecule 24 may be royal blue.

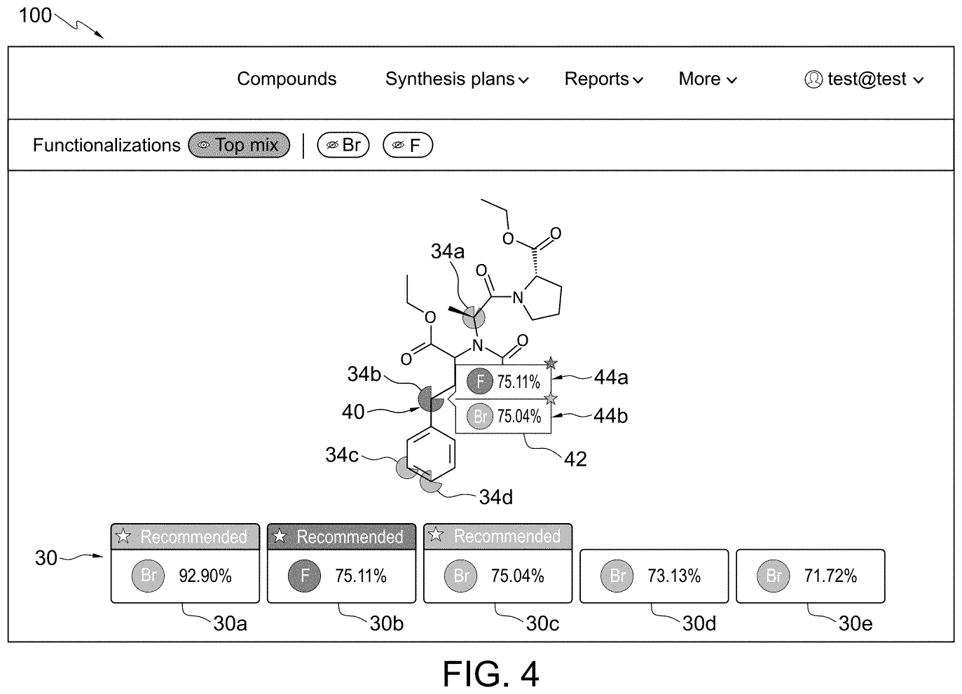

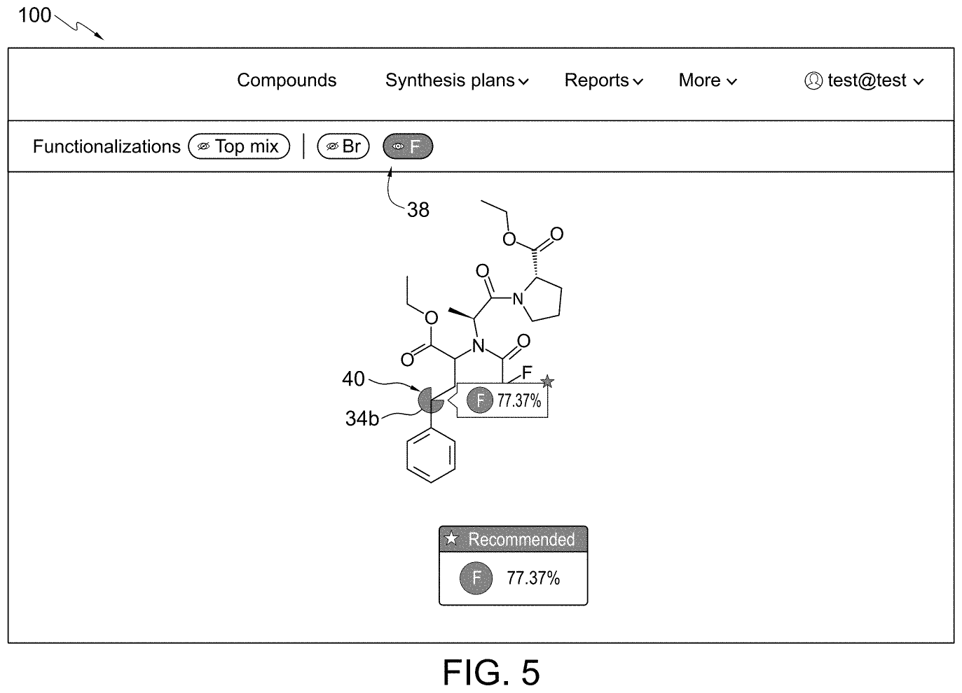

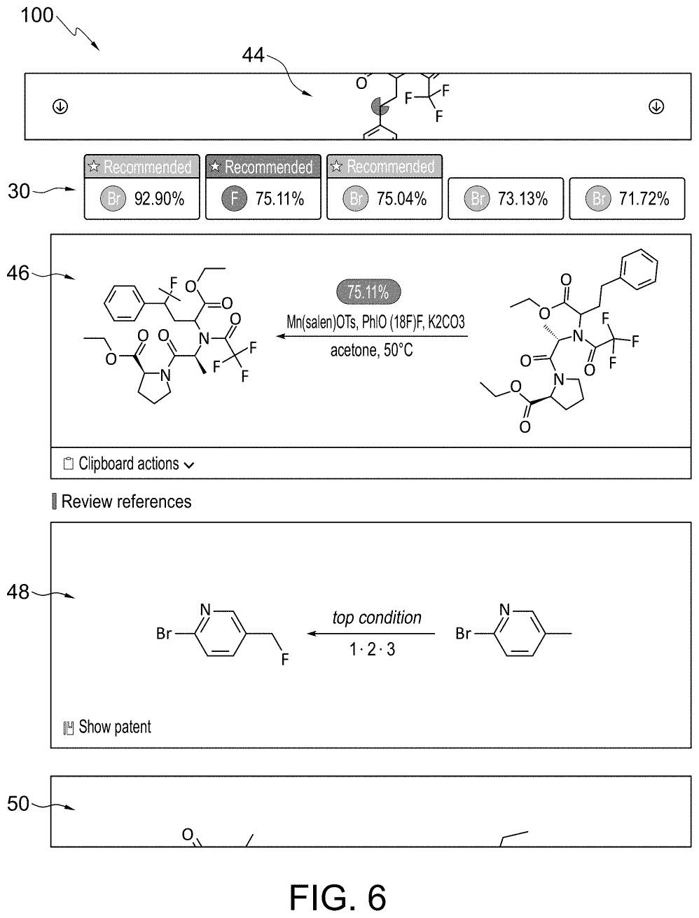

FIG. 3 is a screenshot of the embodiment of a GUI to an embodiment of model for predicting outcomes and conditions of chemical reactions showing the functionalization search exploration overview. A list 30 includes currently visible functionalizations 30a . . . 30e, selecting each of which redirects to the functionalization detail view (FIG. 6), is displayed next to the selected input molecule 32 (molecule 12 in FIG. 1). A number indicating the confidence level of the model prediction (e.g., the percentage shown) for each functionalization 30a . . . 30e may be included. A selection of top predictions is visible by default. Specific locations corresponding to each prediction are marked on the graph with pie charts (34a . . . 34d) which encode the confidence score (value) and the type (color) of the top prediction in each location. As shown, functionalizations correspond to pie charts as follows: 30a and 34a, 30b and 34b, 30c and 34c, 30d and 34d. A pie chart corresponding to functionalization 30e is not shown. Locations may be “hovered” by maintaining an indicator over the item, as shown in FIG. 4, to reveal information. Predictions may be filtered by functionalization type, as shown in FIG. 5. In this screen, like functionalizations may be colored similarly by the associated atoms of the functionalization, e.g., F functionalizations found for molecule 32 may be colored light blue and Br functionalizations found for molecule 32 may atoms may be beige.

FIG. 4 is a screenshot of the embodiment of a GUI to an embodiment of model for predicting outcomes and conditions of chemical reactions showing the exploration overview revealed by hovering over a location 40. The hovering causes a list 42 of functionalizations found for location 40 to be displayed in a floating menu next to the location. Each functionalization 44a, 44b may be selected to view details regarding the functionalization, e.g., the confidence level percentage. In this screen, like functionalizations may be colored similarly by the associated atoms of the functionalization, e.g., F functionalizations found for molecule 32 may be colored light blue and Br functionalizations found for molecule 32 may be beige.

FIG. 5 is a screenshot of the embodiment of a GUI to an embodiment of model for predicting outcomes and conditions of chemical reactions showing the exploration overview results filtered by functionalization type F 38. By selecting functionalization type F 38, the list of functionalizations displayed when hovering over location 40 is reduced to those that satisfy the filter criteria.

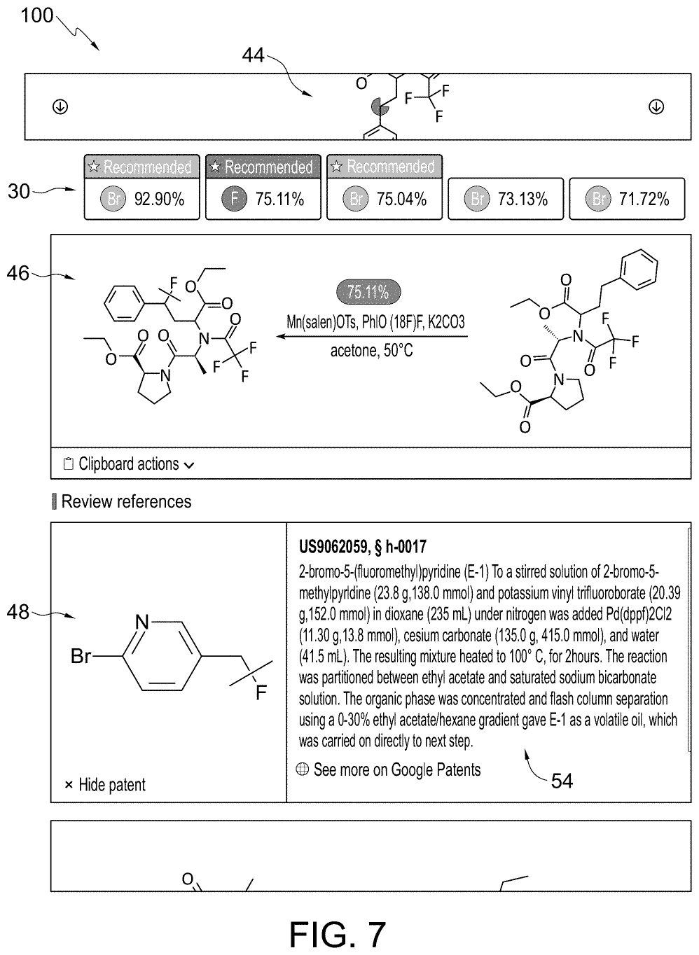

FIG. 6 is a screenshot of the embodiment of a GUI to an embodiment of model for predicting outcomes and conditions of chemical reactions showing the functionalization detail view. The screenshot illustrates that the GUI provides navigation to an overview 44 of the selected location, as well as other functionalizations in the list 30. A predicted reaction graph 46 is displayed that includes substrates (on the right and also associated with the reaction direction arrow) and reaction conditions (associated with the reaction direction arrow). A list of reference reactions 48, 50 (partially displayed) is shown below the predicted reaction. Each reference reaction, e.g., reaction 48, may be further expanded 52 to show detailed information, such as that shown in FIG. 7. In this screen, like functionalizations may be colored similarly by the associated atoms of the functionalization, e.g., F functionalizations may be colored light blue and Br functionalizations may be beige.

FIG. 7 is a screenshot of the embodiment of a GUI to an embodiment of model for predicting outcomes and conditions of chemical reactions showing the functionalization detail expanded for reference reaction 48. In the expansion of link 52 (FIG. 6), a procedure 54 is displayed as a result.

### Section 1

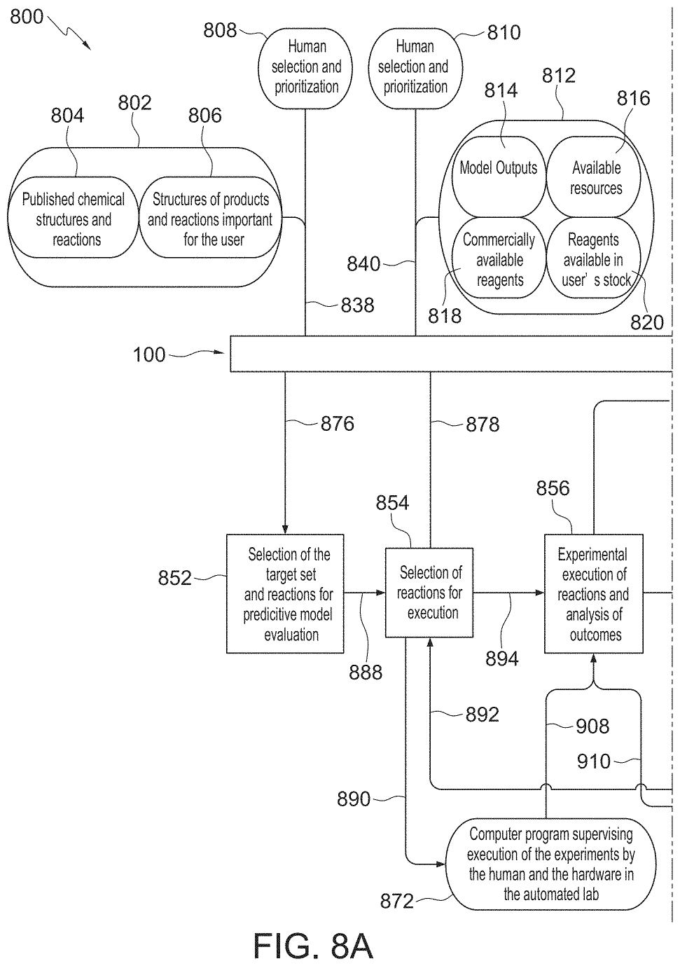

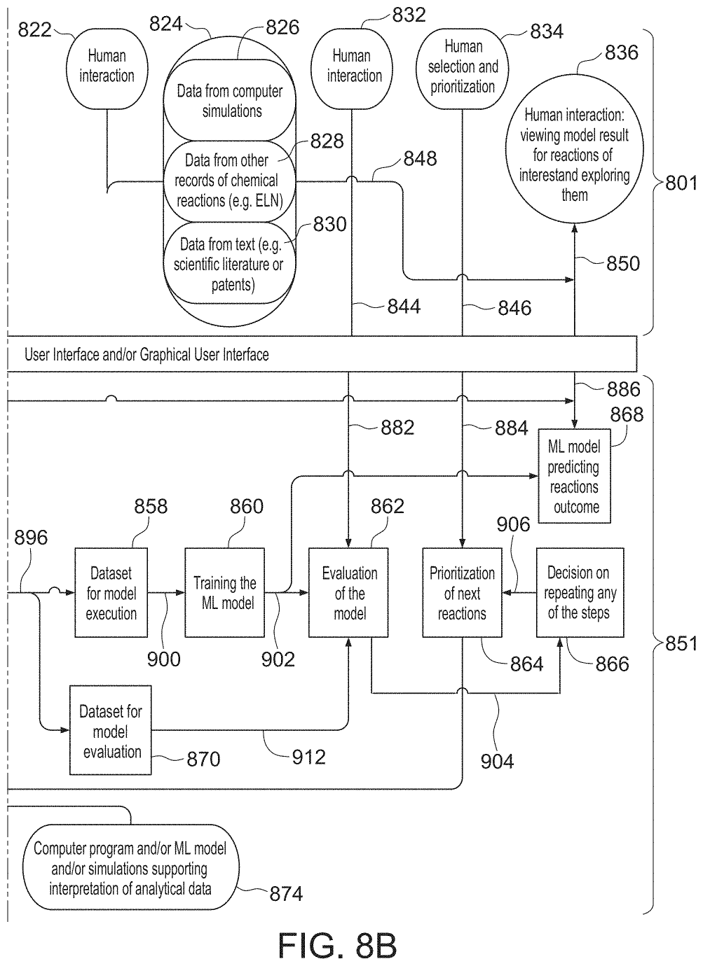

FIG. 8A and FIG. 8B are first and second halves, respectively, of a flowchart illustrating an embodiment of method 800 for predicting outcomes and conditions of chemical reactions, within which is an embodiment of a data collection method. In some embodiments a Data Collection Method or Data Collection may be understood as a method comprising multiple steps (such as selecting the Target Set, or purchasing reagents), used to design and perform experiments results of which used to train the machine learning model that is used by the disclosed system. One of the goals of the Data Collection Method is to collect data that is relevant for making accurate predictions about high yielding reaction conditions for chemical transformations of specific classes in a specific subset of the chemical space.

The method 800 comprises several steps that may (but don't have to) be executed in the depicted order. FIG. 8A and FIG. 8B illustrate action 801 that provides input to or receives output from computer system(s) 851 via GUI 100. The first step 852 is the selection of the Target Set (see text for examples) User input 808 may be received via GUI 100 via path 838, 876. The Target set may be selected from external sources 802 which may include published sources 804 or other structures of products and reactions important for the user 806. The selected Target set is provided 888 to Step 854, which focuses on the prioritization of reactions for execution and may receive user input 810 via path 840, 878. Prioritization input 810 may be selected by the user based on considerations 812, which may include model outputs 814, available resources 816, commercially available reagents 818, and reagents available in user stock 820. In step 894 the selected reactions are forwarded to step 856. The selected reactions are forwarded 890, 908 via step 872 in which a computer program may supervise the execution of the experiments by the user and the hardware in an automated lab. In step 856, reactions selected in step 854 are executed and their outcomes analyzed. The analysis of outcomes in step 856 may in some embodiments be assisted 910 by a step 874 where computer program and/or ML model and/or simulations supporting interpretation of analytical data are performed. The results of step 874 are provided to steps 858 (via path 896), 870 (via path 896), 868 (via path 886) and 836 (via paths 886, 850). In step 858 a dataset for model training is assembled. In step 870, a dataset is assembled for model evaluation 898. The dataset for model training from step 858 is provided via path 900 to a step 860, in which the ML Model is trained. The trained Model is then made available via path 902 to step 862 in which the Model is evaluated. In the evaluation of the Model, step 862 may receive further input from step 870 via path 912 of a dataset for model evaluation. Step 862 may also receive user selection and prioritization input 832 via paths 844, 882. In step 866, a decision is made on whether to repeat any steps. With input from the decision 866 via path 906, and from potential user selection and prioritization input 834 provided via paths 846, 884, a set of next reactions are prioritized in step 864. The prioritized next reactions may be provided via path 892 to step 854 to be included in the list of reactions from which to select for execution. In step 836, the user interacts with the Model to view results of the Model from step 868 for reactions of interest and for exploring those reactions. Both steps 836 and 868 may receive user input 822 via path 848, which may include additional data 824, including, for example, data from computer simulations 826, data from other record of chemical reactions (e.g., ELN) 828. and data from text (e.g., scientific literature or patents).

Fundamentally, as shown in FIG. 8A and FIG. 8B, users can interact with the Model and Dataset, and may supervise the method via dedicated user interfaces, such as graphical user interface (GUI) 100. In some embodiments, the Model or Current Model (which implies a re-training of the Model has occurred) may be understood as the machine learning model trained on all or some of the chemical data available in a given step of the method. For example, a user may use the resulting Model (e.g., at step 836) and a user interface access the Model (at step 868) to predict high yielding conditions for a chemical reaction. The goal of one embodiment of the method, which is to train the Model to provide predictions of reaction outcomes and to predict optimal reaction conditions for fully-specified reactions (see Definitions) at a satisfactory performance (see text for examples of how performance is measured) can be obtained after a single iteration or multiple iterations of the data collection and model training cycles, as discussed within.

In some embodiments, the user may be a computer system that interfaces with the system, e.g., through an API. In some embodiments, there may be both a human user arid a computer system/user. That is, in this discussion, a step performed by a user may be performed by a human user or a computer system user, or either, or both.

In some embodiments, a Target Set may be understood to include a set of chemical reactions (fully or partially specified) that is an input in one of the steps of the Data Collection Method. The set is usually defined by the user. The set can be defined explicitly (e.g. reactions of a specific class that form known drug-like compounds that are either approved or in clinical trials) or implicitly (e.g. reactions of a specific class that form as the product a chemical compound that satisfies the so-called Lipinski Rule of Five, which puts constraints on properties such as molecular weight).

In some embodiments, the Model or Current Model (which implies a re-training of the Model has occurred) may be understood as the machine learning model trained on all or some of the chemical data available at a given step of Data Collection. The model takes as input a partially specified chemical reaction (the reaction can be partially defined in the sense that the input may exclude some of the necessary conditions or other pieces of information, such as outcome). In the case where the model receives as input a reaction that only lacks outcome information, it outputs the outcome of the reaction. In the case where the model receives as input a partially-specified reaction, the model outputs one or more predicted fully-specified reactions. The Model can output any number of additional outputs such as explanations for its predictions or model's certainty about the prediction.

In some embodiments, a Current Dataset may be understood as the dataset available at a given step of Data Collection, relevant for training the Current Model, which may be assembled from already performed experiments and potentially joined with any other available sources of data (e.g. from publicly available literature or based on quantum computation).

In some embodiments, Chemical reaction or reaction may be understood as a fully-specified chemical reaction that includes all information about reactants and conditions, as well as information about the outcome of the reaction. In some embodiments, the outcome of the reaction may be understood as what products are formal in what yields (percentages), which may be understood as a special case as a binary indication whether or not a given product was formal with yield above a given value (dependent on the context, e.g. provided by the user). In particular, a skilled chemist should be able to perform the chemical reaction based on these information in the laboratory.

In some embodiments, Chemical reaction without outcome may be understood as a fully specified chemical reaction missing only the information about the outcome.

In some embodiments, Reaction conditions may be understood as being part of a comprehensive description of the way of performing the fully-specified chemical reaction excluding the structures of the reactants which contribute their atoms to the structure of the expected product (referred to as reactants). For example, reaction conditions include, all physical variables that influence the reaction: temperature, pressure, reaction time, stirring intensity, order of addition of reagents, rate of reagent addition, all components of the reaction mixture (such as solvent(s), catalyst, base, acid, coupling reagent) which do not contribute their atoms to the expected target product quality of the used reagents, proportions of reaction mixture components.

In some embodiments, a partially-specified chemical reaction or a partially-specified reaction may be understood as a partially-specified reaction with an omitted piece of information, which can be any piece of information from a fully specified chemical reaction. In particular, it may have missing conditions such as solvent or catalyst.

In some embodiments, the outcome of a chemical reaction may be understood as the products that are formed and in what yield each product is formed for a chemical reaction.

In some embodiments a computer system is disclosed for predicting chemical reaction conditions that satisfy user requirements (e.g. are high yielding, based on partially specified chemical reactions) and/or predicting outcomes of given chemical reaction that is designed to achieve high accuracy on chemical reactions specified (implicitly or explicitly, as shown in examples) by a user (referred below to as Target Set) thanks to the combination of 1) and, optionally, 2).

1) A machine learning model trained on a diverse set of experimental outcomes of chemical reactions combined of:

(1)(i) Experiments designed and performed with a methodology that combines one or more of the following features, chemical reactions are selected with the end goal of enabling a trained model to achieve high accuracy on the Target Set (see more details on the method for prioritization of chemical reactions in Section 2.2 and Section 5); performed in a high-throughput fashion with the aid of automated liquid and/or solid dispensers (e.g. by means of picking proper methods or experimental hardware, see more details in Section 3 and Section 5); outcomes of reactions (what products are formed and in what yield) are analyzed (run for example through an LCMS machine with UV/Vis detector) and quantified (processed for example by off-the-shelf software to determine yield of the reaction), or involving decisions made by one or more user (see more details in Section 2.4 and Section 5, in particular on which decisions a user may make), or

(1)(ii) Optionally, other sources of chemical information including chemical reactions extracted using any method from textual information such as disclosed patents or scientific literature, or other datasets of chemical reactions including, but not limited to, datasets derived from documented records of executed experiments (such as electronic laboratory notebooks); or

(1)(iii) Optionally, outcomes of computer programs that aim to simulate outcomes of chemical reactions using molecular modeling at various levels of theory (level of theory is a term used in computational chemistry; higher level of theory means that a computer program achieves higher accuracy of simulation at the expense of longer processing time).

Furthermore, the machine learning model may be trained using techniques that enable more accurate predictions on the Target Set, as described later in Section 2.4.

2) Optionally, a computer program with a graphical user interface that unlocks the ability for the user to achieve a desired goal. Embodiments of such a computer program include (i) and (ii):

(2)(i) A computer program that plans synthesis pathways using any known algorithm, and consistent with user-specified constraints (in embodiments, these may be, but are not limited to, available chemical hardware, preferred conditions or limits on price or availability of reactants to purchase). In an embodiment, the computer program may use the machine learning model to predict and show reaction conditions (see also Section 5.3). In one embodiment, the synthesis steps along the computer designed pathways can be executed partially autonomously on one or more pieces of laboratory equipment; in an embodiment, the system may communicate with the laboratory equipment through an application programming interface. In another embodiment, the synthesis pathway planning outcome can be summarized in a single number that indicates the expected cost of synthesis of the compound that is shown to the user.

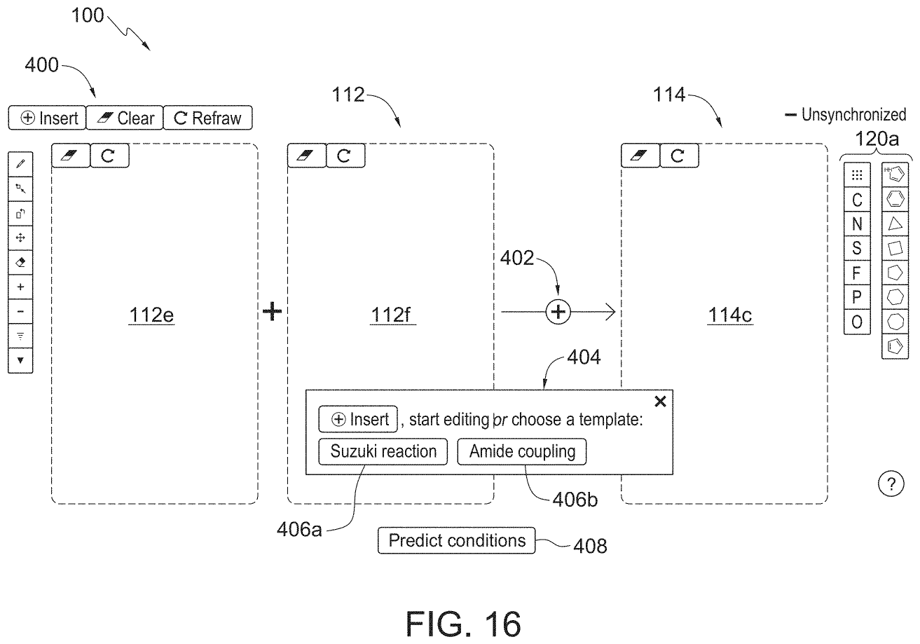

(2)(ii) A computer program that allows the user to find reaction conditions that satisfy user constraints (e.g. are high yielding) for user-specified chemical reactions through a graphical interface that allows the user to receive predicted optimal conditions made using the machine learning model (see FIG. 5 and FIG. 16). In one embodiment of this kind, the user is able to effectively interact with the model predictions (e.g. explore them or modify them ). In one embodiment of this kind, the program allows the user to input only one of the substrates (rather than a full reaction), and the program suggests reaction conditions for synthesis of multiple potential products using the machine learning model. See Section “5.2. Predicting reaction outcomes and the optimal conditions for a selected reaction” for more details. Regarding the user ability to choose constraints on the predictions, this ability allows the user to define, by their choice of constraints, what that user thinks might result in the optimal conditions.

Furthermore, some embodiments provide for the synthesis planning and the potential synthesis of a collection of compounds, where specific examples include: a DNA-encoded library may be synthesized based on predictions made by the system: and a virtual catalog of chemical compounds may be created based on predictions of the system.

### Section 2. Data Collection Method

### Section 2.1

In an embodiment, a Data Collection method involves one or more iterations. Each iteration includes execution of one or more the following steps: 1) selecting a batch of chemical reactions for execution in the laboratory, for example based in part on their similarity to the Target Set (see Section 2.2 and Section 5 for description of the methods for selecting the batch); 2) performing the selected batch of chemical reactions in a laboratory and analyzing the post-reaction mixtures by appropriate analytical method with the goal of quantifying the yield (percentage of product formed) of the reaction (this step may require purchasing chemical matter on the market), 3) estimating the outcomes of the executed reactions using software processing of analytical data; 4) obtaining a new Model by training it on the Current Dataset (that also includes all or a subset of the reactions executed in the previous step), 5) analysis of performance of the model according to a number of metrics (which can be displayed in the GUI, as defined later in the document) and deciding whether or not to continue Data Collection (see Section 2.4 for more details), or 6) interacting with the Model and the Dataset in a user interface (graphical or not). In particular, the interaction includes inputting to the model partially-specified chemical reactions and reading/displaying the output, (predicted outcomes of chemical reactions along with additional information such as reaction conditions) (see Section 5 regarding this step).

### Section 2.2

In one embodiment, the Target Set consists of any set of reactions that are relevant for a given application of the final Model.

In one embodiment, the Target Set reactions are any reactions that their products are molecules that are or have been in clinical trials or were identified as potent binders/inhibitors of relevant biological targets. In one embodiment of this kind, only reactions of a given type (for example amide couplings) are included in the Target Set.

In one embodiment, a machine learning model, and/or a heuristic algorithm and/or user inspection is used to reduce the size of the Target Set by prioritizing certain chemical reactions in order to achieve one or more goals such as lower chemical similarity of reactions in the Target Set to other reactions in the Target Set, appropriate coverage of specific user defined chemical space, with the lowest size of the Target Set.

Regarding selecting chemical reactions for execution, in one embodiment, reactions to be performed, analyzed and added to the Current Dataset at a given step of Data Collection are selected from the space of any possible chemical reactions as the highest scoring set of reactions according to the following mathematical formula of Eqn (1):

S=argmax_{S,|S|=N} f(S), Eqn (1)
where t(S) is a scoring function that assigns score to a set of reactions, and S is a set of N reactions that are part of the batch. We will refer to fin the rest of the document as the Reaction Prioritization Function.

In embodiments, the function f(S) may include a combination of one or more factors defined below: a) Dow many reactions that are part of the Target Set are chemically similar (as defined below) to reactions in the set S; b) How many reactions that are (1) part of the Target Set are chemically similar to reactions in the set S, and (2) are assigned low certainty by the Current Model, c) How many reactions that are part of the Target Set are (1) similar to the reaction set S and (2) deemed unlikely by one or more users (scored below a predefined score according to a predefined scale) (In some embodiments, users can be shown each reaction in a GUI (see Section 2.4))(In some embodiments, expert opinion can be approximated using a machine learning model): d) The price of the reagents needed to perform reactions S, e) Time of arrival to the laboratory of the reagents from a provider of chemical compounds from the day of ordering (In particular, whether or not reagents are already purchased): f) The certainty of the reactions in the set S, as assigned by the Model: g) The similarity of the reaction (e g in the form of Euclidean distance or variance of Model predictions) to the Dataset (used to train the Model); h) The chemical similarity (as defined below) of the set of reactions S to themselves (In one embodiment, the formula takes as input the distribution of chemical similarity between reactions in the set S. Generally, we would often like to have a low similarity of chemical reactions in the set S, as it indicates that they cover a larger subset of the chemical space (and hence likely cover a larger subset of the target set). In other words, they should be similar to the target and dissimilar to each other), i) The uncertainty estimation of the Model (In one embodiment the uncertainty is based on the average of different copies of the Model, each retrained on the same Dataset.); j) The type of the chemical reaction (In one embodiment, only reactions of a given chemical type are selected (e.g. only amide coupling reactions)); and k) a Score assigned by one or more users that reflects the opinion whether or not the reaction will be relevant for improving performance of the Model on the Target Set (In some embodiments, user(s) are shown chemical reactions in the GUI (see Section 2.4)).

In one embodiment, the chemical similarity between reactions used in determining the set S is based on numerical representation of reactants (substrates, product) and reagents in each reaction. In one embodiment, the numerical representation is computed using any publicly available method for representing chemical compounds such as MACCS or Morgan fingerprint, jointly referred to as a chemical fingerprint. In one embodiment, the chemical similarity function is based on a chemical fingerprint computed for a molecule with removed atoms that are further than a particular distance front the reaction center, where the reaction center is defined as the atoms that are affected during the chemical reaction. In one embodiment, the numerical representation is computed by inputting the chemical reaction into the Model and saving its hidden representation of the chemical reaction. In some embodiments, the numerical representation can be used to compute the chemical similarity using a measure of similarity between two sets of numerical representations such as the Euclidean distance or the Jaccard index.

Regarding an optional step of Data Collection, in one embodiment. Data Collection may include a step involving purchasing a large number of reagents to make them immediately available for performing reactions involving them. In particular, if the reaction prioritization function f(S) includes the time availability factor (f), these reactions will be naturally prioritized for execution.

In one embodiment, the reagent set R can be prioritized by finding set of reagents R that maximize the following mathematical formula of Eqn (2):

R=argmax_{R,|S|=N} g(R0, Eqn (2)
where g(R) is a scoring function that assigns score to a set of reagents R, and R is a set of N reactions that are part of the batch. In one embodiment, the function f(R) includes one or more factors including: a) any factor of the following form: Let V denote the set of reactions that can be potentially performed given the set of reagents R (The factor is f(V), where f is the Reaction Prioritization Function); b) the price of the reagents from provider of chemical compounds; c) time of arrival to the laboratory of the reagents from a provider of chemical compounds from the day of ordering (in particular, whether or not reagents are already purchased, in which case the time t=0); or d) the chemical similarity of the set of reagents R to themselves.

In one embodiment the set of reagents R, or the set of reactions S, is picked according to the following iterative optimization algorithm that aims to find an approximate solution to the optimization problem posed in (2). In a first step, each reagent or reaction is individually scored if f(S) (g(R) can be decomposed as f(S)=\sum f(s_i) (g(R)=\sum g(r_i)). In a second step, one or more reagents or reactions with the highest scores are picked. The first and second steps are iterated until the desired number of reagents or reactions (N) is selected. The desired number N is a parameter set by a user of the method, and can be different in different steps of the method.

In one embodiment, the solutions to equation (1) and (2) may be solved by using any off-the-shelf software for discrete optimization.

In one embodiment, one or more users can be shown in a graphical user interface different sets of reagents R of reactions S and asked for additional input, which can be used as part of the scoring function f(S) or g(R).

### Section 2.3

Some embodiments include using data from other sources. In one embodiment, the Current Dataset includes chemical reactions extracted front textual information such as academic journal articles or patents. In one embodiment of this kind, the extraction can be done automatically using a machine learning model that is trained to automatically extract chemical information from text data. In one embodiment of the kind, the machine learning model is first trained in a self-supervised manner (using popular pretext tasks such as predicting the next word in the sequence) on the text it will be using to extract chemical information from. In one embodiment of this kind, the Transformer architecture is used to perform the extraction. In one embodiment of this kind, the following computational pipeline is used to extract information from textual data: (i) predicting (using the Transformer architecture) whether a fragment of text describes a chemical reaction, (ii) labeling (using the Transformer architecture), within a chemical reaction description, paragraphs as headers, descriptions, or footers, (iii) predicting (using the Transformer architecture) from a reaction description, entities such as reaction product, substrates, solvents, catalysts, and other conditions necessary to perform the reaction.

In one embodiment, the Current Dataset includes auxiliary sources that are not directly related to predicting outcomes of chemical reactions. In one embodiment of this kind, a dataset of molecular properties (of any kind and computed using any means such as quantum chemistry computation) is joined with the Dataset. In one embodiment of this kind, the dataset with auxiliary sources of information is picked based both on a similarity to the Target Set and any type of chemical similarity to reactions in the Current Dataset.

In one embodiment, a quantum computer or quantum chemical computation program can be executed to predict (approximate) outcomes of any chemical reactions, instead of or in parallel to high throughput experimentation, in order to enrich the dataset with additional data. In one embodiment of this kind, the same procedure as used to select reactions for execution in the laboratory, can be used to select reactions to be predicted using the quantum computer or quantum chemical computation program.

In an embodiment, a method for creating more accurate machine learning models for predicting properties of compounds and reactions based on quantum computation is disclosed. The embodiment is based on the premise that (a) accurate simulations of simplified quantum systems, where the term “simple” can refer both to the simplification of reaction mechanisms and/or reagents involved in a chemical reaction, can be obtained within reasonable computational budget, and (b) machine learning models may strongly benefit from access to data for simplified quantum systems when making predictions on more complex data, such as experimental reaction outcome data or simulation outcomes of more complex quantum systems. In some embodiments of this kind, one or more computational pipelines are established (as described in the next paragraph) to compute outcomes of chemical reactions for a broad range of different substrates and products, which is then used (as one of elements) to train a machine learning model. In one embodiment of this kind, multiple quantum chemical computational pipelines are established that each aim to contain specific information about one aspect of a chemical reaction. By using the pipeline to compute outcomes of any chemical reactions, the Dataset can be enriched with relatively accurate simulated reaction outcomes. This technique can be particularly useful for chemical compounds that are not covered well in the experimental datasets.

A quantum computational pipeline can be parametrized in a large number of ways such as: (i) the algorithm used for computing energy of a molecule (such as GFN-xTB), (ii) the transitions state of the reaction being simulated, (iii) parameters of the algorithm used for computing energy of a molecule (such as the number of molecular orbitals to use, the error tolerance of the algorithm).

In one embodiment of this kind, a quantum chemical computational pipeline is established, by searching through different possible parametrizations, that achieves significant correlation with experimental data for a given subset of chemical compounds (e.g. smaller compounds). In one embodiment, the Dataset includes chemical reactions with outcomes that are computed according to the quantum computation methodology described in the previous paragraph.

### Section 2.4

### Section 2.4.1: Asking User(s) for Input During Data Collection

In various embodiments. Data Collection may include asking user(s) questions with the goal of using the answers to steer the process. In some embodiments, more than one user may be asked the same question, and the answer is pooled from all users using a method appropriate for the context (for example using maximum voting in the context of deciding whether or not to stop Data Collection).

In some embodiments, user(s) can be asked one or more of the following questions (described here only briefly and expanded on in other places in the document): a) whether to continue Data Collection (see Section 2.4.3 for more details); b) to rate on some scale the relevance of a chemical reaction or a set of chemical reactions for improving performance on the Target Set (see Section 2.2 for more details), or c) to specify different parameters of the Data Collection, which may include but are not limited to: (i) the number of reactions to prioritize, (ii) what is the Target Set, or (iii) what function to use during reaction prioritization (see Section 2.2). Details about what specifically are the parameters are included in the relevant Sections.

In some embodiments, a machine learning model can be used to predict answers given by users by training the answer-predicting model on a dataset of collected answers in prior iterations or executions of Data Collection in some embodiments, the answer-predicting model can be based on the Transformer architecture with the input consisting of a sequence of tokens that represent relevant context of the question (such as history of the Data Collection method) and output is a sequence of tokens representing the answer (such as “0” or “1” indicating whether to stop or continue Data Collection).

### Section 2.4.2: Supporting User(s) in Answering Questions

In some embodiments, user(s) can use a dedicated user interface (such as a graphical user interface (GUI) to help them answer more accurately questions raised during Data Collection such as making the decision whether or not to continue Data Collection. See Section 2.4.1 for more details about what questions might be raised. The text below describes features of the GUI.

In some embodiments, the GUI has features that can be used in any Use case as discussed in Section 5. In particular, and less formally, the GUI can support querying the underlying Dataset, asking the Model for prediction, viewing user-interpretable explanations (such as Scientific Arguments, see Section 5 for more details) for Model predictions.

In some embodiments, each Current Model performance is summarized (according to one or more metrics discussed in Section 2.4.3) and displayed in the GUI.

In some embodiments, a user can execute queries against the Database using the GUT to support answering a question in any step of Data Collection. The queries can be specified by any number of means, including: (a) the presence or absence of given chemical substructures, (b) a similarity to a given chemical reaction, (c) the presence or absence or value of given chemical properties (e.g. lipophilicity or acidity). Executing the queries can enable making better choices thanks to better understanding of chemical reactivity. In one embodiment of this kind, the GUI can be used to help answer the question of the likelihood that the Model correctly predicts yield of a chemical reaction. In one embodiment, the graphical interface used to explore the Dataset that is disclosed as part of Use cases (see Section 5) can be also used in the Data Collection process for the purposes of querying the dataset.

### Section 2.4.3: Evaluation and Decision Regarding Whether or not to Stop Data Collection

In some embodiments, the Data Collection process includes a step in which a decision is made about whether or not to continue the process. In some embodiments, the decision is made in part or fully by users. In some embodiments, the decision is made fully autonomously.

In some embodiments, the decision is based on evaluating the performance of the Model (e.g. its ability to predict outcomes of chemical reactions) by summarizing it in one or more metrics. In some embodiments, the metrics may include (i) the accuracy of the Model in discriminating high from low yielding reactions (the percentage threshold that separates high from low yielding reactions can be determined by the user), (ii) the accuracy of the Model in predicting fully-specified reactions based on partially specified reactions; (iii) the correlation between the predicted yield (yield is part of the outcome of reaction) and actual yield for a selected product (for example the highest yielding product). In embodiments of this kind where the decision is fully autonomous, the user specifies a set of logical constraints based on user metrics that, when satisfied, terminate the process of Data Collection (a user is asked to stop Data Collection or a computer system driving data collection checks the satisfaction of the constraint and stops Data Collection).

In some embodiments, the metrics can be computed on reactions (referred later to as an “evaluation set of chemical reactions”) coming from one or more sources, (a) the Target Set, (b) performed reactions that were not used to train the Model, (c) a separate set of reactions that are selected and performed specifically for the purpose of evaluating performance by a user or autonomously using any method (for example this set may include a set of particularly challenging reactions that was not included in the Target Set). In one embodiment of this kind, the reagents that are part of the evaluation set are more chemically complex than reagents included in the experiments (where the chemical complexity, for example, is gauged by the number of different chemical substructures from a predefined fist). In another embodiment of this kind, the reagents in the evaluation set can be selected using a method that selects most similar reactions to known drugs or compounds in clinical trials in terms of chemical similarity (as discussed before) between reactants of the reactions to known drugs or compounds in clinical trials.

### Section 2.5

### Section 2.5.1: Features of the Model

In some embodiments, the Model can be trained (where the method to train the model depends on the exact type of Model used, see Section 2.5.2 for more details) to have one or more of the following features: a) to be able to predict the outcome of a reaction based on input consisting of a fully-specified chemical reaction (in which case the Model masks the specified outcome), or a partially-specified chemical reaction; and b) to be able predict one or more chemical reactions based on a fully- or partially-specified chemical reaction, which can include additional pieces of information (for example information about the used conditions). A fully-specified chemical reaction is one that includes all information about reactants and conditions, as well as information about the outcome of the reaction (what products are formed in what yield (percentages)). In particular, a skilled chemist should be able to perform the chemical reaction based on this information in the laboratory. Partially-specified chemical reaction or partially-specified reaction; a partially-specified reaction has a piece of information omitted from a fully specified chemical reaction. In particular, it might be missing information regarding conditions such as solvent or catalyst. A concrete example of (b) is predicting high yield reaction conditions based on input consisting of substrates and products.

In some embodiments, the Model outputs can include the uncertainty about its predictions (details about the method of computation are in the next paragraph). Model uncertainty can be used to improve the accuracy of predictions at each stage of Data Collection where the Model is used, e.g., when the Model is used (a) in reaction prioritization during Data Collection (see Section 2.2), or (b) an additional output shown in GUI in any Use case (such as displaying predicted reaction conditions).

In some embodiments, the Model input may include a set of user requirements for generated chemical reactions with additional information such as conditions. In some embodiments, user requirements include one or more of the following type;
(a) the reaction has the highest possible yield;
(b) the reaction conditions satisfy certain constraint such as using low temperature;
(c) the substrates and products satisfy certain logical constraints, such as being below a given purchase price.

In some embodiments, an ensemble of Models can be trained, and the Model can be understood as consisting of multiple variants of the Model. In some embodiments, the individual variants of the Model can be obtained by repeating the training procedure but changing the configuration in some more minor or more important ways, such as changing the order of training examples in which they are shown during training, or using different parameters of the training procedure (such as the length of training). In these embodiments, when the ensembled Model is asked for providing output, the input is provided to each variant, and the outputs are pooled according to any method such as averaging the outputs (in cases averaging is well defined) or voting (in case the output is categorical).

In some embodiments, the Model outputs can further include outputs that are geared towards increasing the interpretability of the Model by users. In some embodiments, the Model can include a list of chemical reactions (fully or partially specified) from the Dataset, which can help a user to form an opinion whether or not the Model output is correct (whether the Model output would agree with experiment) by having the ability to view outcomes of already performed reactions related to the predicted reaction. In some embodiments, the Model can include a user-interpretable explanation for why it made the prediction, such as: (a) outputting a prediction about physiochemical properties that are relevant for the prediction (e.g. solubility of the product in water), (b) including prediction of the reaction mechanism (e.g. showing the critical transition state along with predicted energy of the transition state). In some embodiments, users may be asked (e.g. via GUI, see Section 2.5) questions related to how convincing or useful are the provided explanations. In some embodiments of this kind, the Model may be trained on a Dataset augmented with information about the provided information on how convincing or useful the provided explanations are.

In some embodiments, the uncertainty can be computed as a confidence interval indicating what is likely the maximum and minimum value of the predicted quantity. In one embodiment, the mean of predictions made by members of the ensemble (in the case of classification output such as predicting whether the yield is above some user-defined threshold or not) or variance of predictions (in the case of regression output such as predicting the yield) is used as a factor influencing Model uncertainty. In another embodiment of this kind, a form of distance (e.g. the Euclidean distance between hidden representation obtained from the model, in the case that the Model is a neural network) between the input and examples from the training set is used in the formula to compute Model's uncertainty.

In some embodiments, the Model outputs separately from the uncertainty estimation a scalar that quantifies whether or not the model was trained on similar data. In one embodiment of this kind, the scalar quantity is computed by training an ensemble of different models and computing the variance of predictions of each member of the ensemble. In some embodiments, the scalar is used to modify the uncertainty estimation in the manner that if the scalar value is low, then the uncertainty is accordingly increased. The scalar quantity can be used in any step of Data Collection where the uncertainty on Model outputs is used (as specified in appropriate places in text).

In some embodiments, the Model output may include a scalar that approximates how a user would judge the associated predicted reaction in terms of how likely the user believes the reaction would have yield above a certain threshold (e.g. on a scale from 1 to 5 that the reaction would achieve a higher yield than 5% of the desired product). In some embodiments of this kind, the Dataset is augmented to include reactions with such assigned scalars, which enables training the Model to predict the scalar.

Multi-task learning is a broad set of techniques enabling training a given machine learning model against a number of tasks such as both predicting what object is in the image and predicting where the object is in the image. Training including a task is understood as configuring training so that the Model achieves a given functionality (task) on a given set of examples. In one embodiment, the Model is trained on the Dataset using any form of multi-task learning. In one embodiment of this kind, individual weights are assigned to subsets of the Dataset. In another embodiment of this kind, the Model is first trained on the full Dataset, and then trained again on a subset of the Dataset.

In one embodiment, the Model or the training procedure may be modified so that the Model predicts reactions that satisfy certain logical conditions, such as temperature being in some specific range, in one embodiment, the Model can be trained or fine-tuned (after training on all reactions) on a subset of the Dataset consisting of reactions that satisfy the constraint. This feature is useful for certain Use cases such as use in the context of predicting and executing syntheses using an automated laboratory. In an automated laboratory, one can potentially use only certain reaction conditions (such as only specific conditions or only specific ranges of temperature). In some embodiments, this can be achieved by adding an additional filtering step added after generating outputs from the Model that excludes outputs of the Model that do not conform to a given logical constraint.

Achieving high accuracy on reactions that have low chemical similarity to reactions in the Dataset might be critical for some Use cases. In particular, drug-like compounds are expensive to purchase because they are often chemically complex (have for example a large number of atoms). Hence, they might be less frequently prioritized during Data Collection (see Section 2.2). In some embodiments, the Model or training procedure can be configured with the goal of increasing accuracy on such reactions in the following ways. In some embodiments, the Model can be created using ensembling (as described in previous paragraph). In some embodiments, training procedure may include contrastive learning, self-supervised learning or semi-supervised learning tasks (these are well-known broad categories of tasks that can be used within the framework of multi-task learning, see previous paragraph) such as predicting removed or masked out parts of the input (for example predicting the substrates based on a reaction where the substrates are removed or masked out). In some embodiments, training can include predicting one or more properties of a molecule (that can be part of some reaction in the Dataset or coming from a different source of data such as the CheMBL database) such as its solubility or boiling point. In some embodiments, training procedures may include tasks or methods geared towards learning a representation (in Models that have an internal representation such as neural networks) that are causally predicting the reaction outcome. In some embodiments of this kind, any method of causal discovery can be applied to discover parts of representation that predict in a more causal manner the outcome of a reaction.

In one embodiment, the Model may be trained using training methods that use meta-information about a given chemical reaction, such as whether or not the reaction outcome was measured in the laboratory or simulated using a quantum computation pipeline.

In some embodiments, the Model input may additionally consist of auxiliary information related to the chemical reaction such as the value of different molecular properties (for example electronegativity of each atom). In some embodiments, these molecular properties can be computed using a quantum simulation software such as ORCA or Schrodinger. In some embodiments, these molecular properties can be predicted using a machine learning model that was trained on a dataset including molecular properties.

### Section 2.5.2: Embodiment Based on the Transformer or Graph-Neural Network Architecture

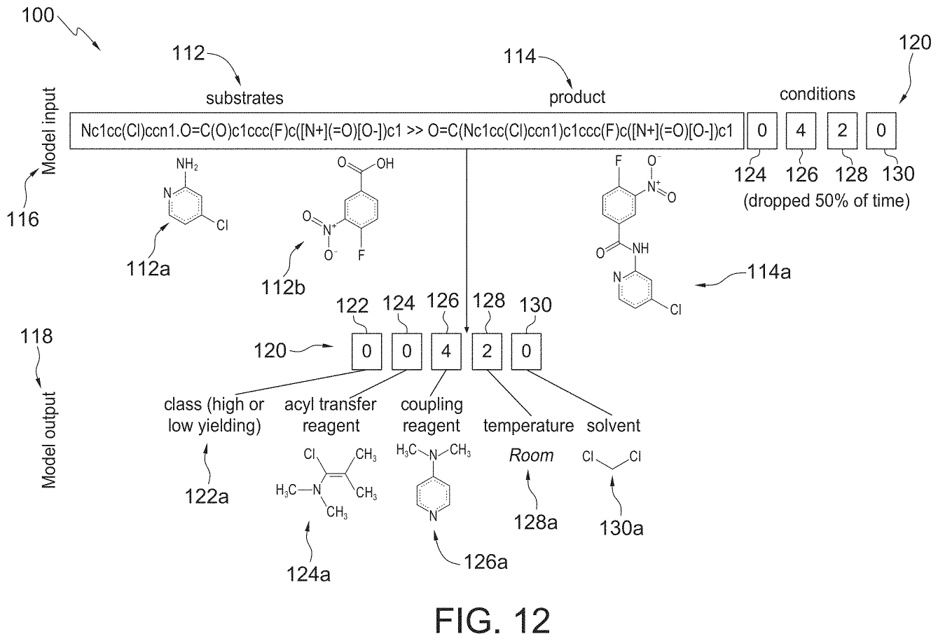

In one embodiment, the Model is based on a sequence to sequence deep neural network such as the Transformer architecture in which both inputs and outputs include a sequence of tokens, where each token has an assigned chemical meaning. In one embodiment of this kind, the substrates and products of the reactions are encoded in the form of a sequence of characters (for example according to the SMITHS notation) and the output is encoded in the form of tokens representing predicted yield and/or reaction conditions. In another embodiment of this kind, the input consists of the reaction with one or more pieces of information of a chemical reaction missing (e.g. missing products), and the output consists of the yield and a prediction of the missing information (for example what should be the products). In some embodiments, the Model can include as output a token indicating whether or not the reaction yield is above a certain (user-specified) threshold. Visualization of the model input and output representation (for amide coupling reaction) is depicted in FIG. 12.

In one embodiment, the Model is based on a graph-neural network, a type of neural network that takes as input a graph with vertices (atoms) and edges (chemical bonds), where each vertex and edge may have additional properties (such as type of atom). The output may be the same as described in previous paragraphs. In some embodiments, reaction conditions are encoded as properties of an additional vertex in the input graph. In some embodiments, each reaction condition is treated as an additional vertex in the input graph.

FIG. 12 illustrates forms of input and output in an embodiment of a GUI 100 to an embodiment of the Model for predicting outcomes and conditions of chemical reactions. In FIG. 12, the Model takes as input 116 substrates 112, specifically 112a, 112b, the product 114, specifically 114a and optionally reaction conditions 120 encoded in the form of one hot encoding 124, 126, 128, 130, or another textual encoding of molecules. The Model outputs 118 encoded conditions 120 including the predicted class 122 (high vs low yielding for some user-defined threshold of yield) as the first token along with conditions 124, 126, 128, 130 (if they were not passed as input) as a sequence of four tokens. In some embodiments, in training, some percentage of training time, parts of input 116 may be masked or removed.

### Section 3. High-Throughput Laboratory

In some embodiments, the Model is trained on the Dataset that contains reactions executed in a laboratory that were specifically designed to increase performance of the Model on a desired Target Set.

In one embodiment, the high-throughput laboratory includes using medium- and high-throughput analytical techniques such as MALDI-MS, Echo-MS, MISER chromatography applied for analysis of composition of post-reaction mixtures in order to determine the quantity of product(s) formed in the reaction and level of consumption of starting material.

In one embodiment, a machine learning model can be used to predict yield of chemical reactions from raw analytical data. In particular, a machine learning model can be used to predict the yield of reactions based on outputs of high-throughput but higher noise analytical techniques such as MALDI-MS, Echo-MS, chromatography in MISER mode. In one embodiment, the model can be trained on a dataset of chemical reactions with quantified yield (using potentially lower-throughput technique).

In another embodiment of this kind, a machine learning model can be trained and used to directly determine the yield of reaction (the quantity of the product) from raw analytical data coming front any analytical device (such as an LCMS machine), which in particular may enable quantification without knowing or measuring the level of analytical signal for the known amount of the pure compound being analyzed (i.e. without knowing the molar absorptivity).

In one embodiment, data coming from LCMS analysis of the post-reaction mixtures can be used to estimate the quantities of selected components of the post-reaction mixture (and recalculated into yield of executed reactions).

In one embodiment, an automation solution can be used to create the reaction mixtures, and for transferring reaction mixtures between different pieces of equipment. In one embodiment of this kind, laboratory hardware such as an automated liquid handler (e.g. Opentron OT-2) or 96-channel pipette (e.g. Integra Mini) can be used to automate creating reaction mixtures (e.g. by automating pipetting).

In an embodiment, a DNA-encoded library (DEL) can be used as means for generating experimental data (outcomes of chemical reactions) on reactivity of DNA-tagged reagents, which may be relevant for training machine learning models (and the Model in particular). In one embodiment of this kind, a library of reagents bearing a common functional group, each tagged with a different DNA tag is used. A mixture of such tagged library components (A) is allowed to undergo a chemical reaction with (a) certain reagent(s) (B), which results in the formation of covalent bonds between some elements of A and some elements of B. Proper construction of reagent B (tagging or immobilization) enables subsequent cheap and reliable isolation and identification of formed A-B adducts. In an embodiment, the reagent B may be attached to a large molecule such a DNA strand, protein (polypeptide), nano-particle, or polymeric resin bead, which enables washing out the unreacted library components A and subsequent identification of DNA tags of the components of library A that underwent the reaction with reagent B, using widely known techniques, such as polymerase chain reaction (PCR) and next generation sequencing (NGS).

In another embodiment of a DEL used as means for generating data, the DEL may be exposed to a molecule that will serve as a binding target for the fragment of interest attached to some molecules within the DEL. The target molecule may be a protein or a small molecule, which may be covalently bound to a solid support material. The molecules that have not bound to the target can be then washed away. In one embodiment, the remaining molecules being pan of the DEL may be identified using generally known techniques, such as polymerase chain reaction (PCR) and next generation sequencing (NGS).

### Section 4. Embodiment of Data Collection Method

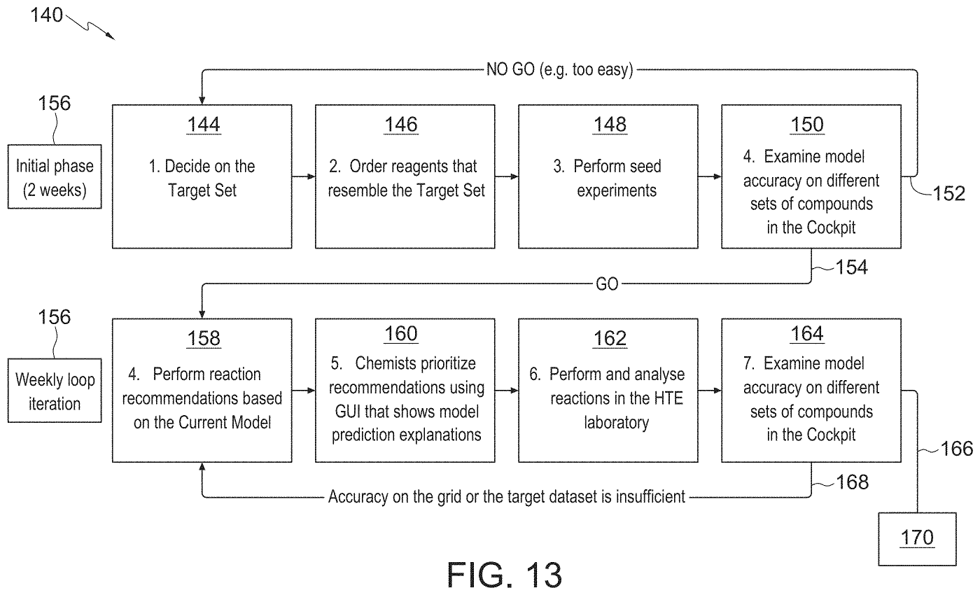

FIG. 13 is a flowchart illustrating an embodiment of a data collection method 140 for a model for predicting outcomes and conditions of chemical reactions. In FIG. 13, the process starts with an initial phase 142, including steps 144-150. Step 144 is the selection of the Target Set. In embodiments, the Target Set may be configured to be composed of reactions whose targets contain a plurality of publicly disclosed drug-like compounds that were identified as potent binders or inhibitors of the recognized biological targets, or are in, or after clinical trials. In embodiments, the Target Set can be based on any subset of such compounds. In step 146, a single large batch of reagents is purchased or otherwise accessed based on their similarity to the reagents in the Target Set and their chemical similarity to other reagents in the Target Set. In step 148, a number of randomly selected reactions involving reagents ordered in the previous step is performed (it is usually impractical to perform all of the reactions involving the purchased reagents because there are too many potential reactions). In an embodiment, the initial phase concludes in step 150 with users examining performance of the Current Model on different sets of compounds (including, but not limited to drug-like compounds that are not part of the reagents mentioned in 1.b above). Based on these examinations, one or more users make a decision whether the Data Collection Method should continue with another iteration 152 of the initial phase 142, or not 154. If not, a subsequent phase 156 is entered. In each (e.g. bi-weekly) iteration of phase 156, steps 158-164 are repeated. In step 158, a number of outputs of the Current Model (with input being reactions involving reagents purchased in previous steps) is computed that can be used in reaction prioritization (see text later). In step 160, optionally the user is asked questions pertaining to which reactions (from reactions involving reagents purchased in previous steps) should be prioritized, in the GUI. In step 162, after determining the final set of prioritized reactions, the prioritized reactions are executed in the laboratory and quantified (i.e. the yield of the reactions is computed based on reaction mixture analysis). In step 164, the Current Model is retrained on the Current Dataset that includes at least in part data generated in step 162, and users examine the performance of the Current Model in order to make the decision whether or not Data Collection should be continued 160 or not 166. The examination may include using the GUI to examine model accuracy on different sets of compounds (see text on evaluation sets of reactions). If not 166, the Model may go on to be employed 170 as described in any of the various embodiments.

In an embodiment, the compounds for step 146 may be purchased from an external provider of chemical matter such as MolPort according to a function g(R), where R is the set of reagents, with the following properties:

g(R) can be decomposed as g(R)=\sum_{i=1}\hat{ }N g(R_i), where R_i is a single reagent to be purchased, and N is the desired number of reagents.

g(R_i) is set to minus infinite value if the compound (R_i) price is above a given threshold or time to arrival is above a given threshold. Otherwise, g(R_i) is set to the number of reactions from the Target Set such that the similarity between one of the substrates and the reagent is above a user-defined threshold

In another embodiment of this kind, g(R_i) can additionally include a term indicating the answer of one or more users how much (for example on scale from 1 to 10) reactions involving the reagent R_i will improve performance of the result Model on the Target Set. As discussed in Section 2.4, in some embodiments, users may have access to the GUI when answering the question.

In some embodiments of this kind, the function g(R) is optimized using the iterative optimization algorithm disclosed before (in Section “2 Data Collection Method”).

In some embodiments of this kind, the decisions whether or not the Data Collection should be continued is based on the performance of the model predicting outcomes of reactions from the Target Set, which can be displayed in the GUI (see Section 2.4).

In the above embodiment, the reactions may be performed in a high-throughput chemical laboratory optimized for achieving a high throughput (number of reactions performed and analyzed per unit of time) and low cost of operation.

In one embodiment of this kind reaction mixtures are prepared in separate wells of multi-well plates of standard size.

In one embodiment of this kind all solutions of all reactants are prepared and stored in separate wells of multi-well plates of standard size, which act as stock solutions for preparation of reaction mixtures.

In one embodiment of this kind, automated liquid handlers with a single-channel or 8-channel pipettes such as the Opentrons OT-2 can be used in one or more stages of the reaction mixture preparation.

In one embodiment of this kind, 96-channel pipettes (such as Integra Mini-96) or 384-channel pipettes can be used in one or more stages of preparation of the reaction mixtures, or post-reaction analytical samples.

In one embodiment of this kind, the multiwell plate containing reaction mixtures is sealed with adhesive polymeric or metal cover or with a silicone or rubber mat. The sealing mats can be held in the correct position by placing the plate with the sealing mat between two rigid panels (one under the plate, the other over the mat) and compressing the panels for example with screws. The reaction mixtures in the wells of the plates can be stirred by shaking the plates in an orbital (thermo)shaker or by magnetic stirring bars placed in each w ell and forced to move by changing magnetic field generated by an external device. The reaction mixtures in the wells of the plates can be heated or cooled by placing the multiwell plates in a thermoshaker or in a heating/cooling block.

In one embodiment of this kind, a known amount of one or more chemically inert compounds is added to selected or all post-reaction mixtures to act as internal standards supporting quantification of the post-reaction mixtures. In one embodiment different internal standards or their mixtures are added to selected subsets of the post-reaction mixtures.

In one embodiment of this kind, multiwell filtration plates with either inert membrane or stationary phase capable of selective absorption of selected components of post-reaction mixture can be used in one or more stages of preparation of post-reaction analytical samples.

In embodiments, the post-reaction mixtures may be analyzed and quantified using off-the-shelf equipment such as the high pressure liquid chromatography (HPLC) combined with one or more detectors, including: single or multi-wavelength UV-Vis detectors; fluorescence detectors; evaporative light scattering detectors (ELSD); charged aerosol detectors (CAD); radiometric detectors; electrochemical detectors; chemiluminescent nitrogen detectors; or mass spectrometers.

In one embodiment of this kind, a pre- or post-column derivatization is applied in the analysis of all or selected analytical samples. Different methods of pre- and post-column derivatization can be applied for various subsets of the analytical samples.

In one embodiment of this kind, the post-reaction analytical samples are analyzed by MALDI-MS, or Echo-MS analytical methods.

In one embodiment of this kind, an aliquot of the post-reaction mixtures is subjected to liquid chromatography and the fraction containing the isolated product in satisfactory purity is collected either manually or with the use of automated fraction collector The amount of the product in the collected fraction is measured by weighing the solid residue after evaporating the eluent(s). In one embodiment of this kind the flow of eluate leaving the column is split with a known split ratio between the fraction collector and the sample destroying detector such as MS or ELSD. In one embodiment of this kind a quartz crystal microbalance is used to assess the mass of the solid residue.

In one embodiment of this kind, the post-reaction analytical samples are analyzed by nuclear magnetic resonance (NMR) spectroscopy. In one embodiment of this kind, the reaction is performed in a deuterated solvent or a mixture of thereof and the product is quantified by NMR in the unprocessed or processed post-reaction mixture.

In one embodiment of this kind, the execution of a selected batch of chemical reactions (including preparation of analytical sample) is supported by a dedicated software. The software uses as an input, among others, the batch of chemical reactions to be executed and can perform any combination of actions listed below.
a) Dividing of the batch of reactions into subsets—each subset executed in wells on a single plate (or group of vessels in a single rack)—in order to optimize the process of dispensing the reagents in the wells (vessels);
b) Assigning each reaction a specific location of a well on a plate (or vessel in the rack) in order to optimize the process of dispensing the reagents in the wells (vessels);
c) Provide human lab operator(s) with a detailed list of steps required for execution of the batch of reactions,
d) Supervise the execution of the experimental protocol by supervising in an interactive way the consecutive step carried out by human(s) and/or laboratory hardware,
e) Generate sets of commands for one or more automated laboratory devices
f) Generate a detailed report on the procedures used to execute the reactions in particular based on monitoring and recording: temperature, pressure, humidity, oxygen (or any other relevant gas) concentration(s), stirring time and intensity, time of start and end of each procedure
g) Generate an output used by the analytical device(s) and/or by the software used to process the raw analytical data

In one embodiment of this kind, during step 164 involving making the decision whether or not to continue Data Collection, the accuracy of predictions made by the Current Model is evaluated on a combination of one or more of the following set of reactions with known outcomes, (i) a random subset of reactions that involves any purchased reagents in the previous steps (ii) a random subset of reactions involving reagents from a smaller set predetermined at the beginning of Data Collection; (iii) a number of reactions that form drug-like compounds. The results of such evaluations can be shown in the GUI. Furthermore, during Data Collection no reactions from these three sets can be used in training of the Model to ensure that the evaluation process meaningfully tests the model in a setting where it passed as input a previously unseen reaction. In the above embodiment, users are shown the computed accuracy in the GUI and asked to make a decision on whether or not the Data Collection process should continue, at the end of each phase.

In an embodiment of this kind, the Current Dataset includes some of the reactions performed thus far during the Data Collection phase. In another embodiment of this kind, the Current Dataset may be joined with reactions extracted from published patents and patent applications.

In an embodiment of this kind, the Current Model may be based on the Transformer architecture (as disclosed in the previous parts of this disclosure).

In an embodiment of this kind, the reaction recommendations may be prioritized during Data Collection according to the following three variants of a prioritization function f(S): (i) f(S) is a random number, which results in a random selection of reactions possible to perform using purchased reagents, (ii) f(S) is a weighted sum of a measure of uncertainty of the Model on the set S and a measure of chemical similarity of the set S, which results in selection of the most uncertain reactions that are chemically diverse, or (iii) in addition to the construction of f(S) described in (ii), the function includes an additional factor that measures the chemical similarity of the products in the reaction set S to the products in the Target Set. In an embodiment, the reaction recommendations may be examined by one or more users using the GUI to narrow down the set S to a smaller number of reactions.

### Section 5. Example Use Cases

In some use cases, the GUI or UI greatly enhances the user's ability to access the Model and thereby achieve a desired goal. However, while this disclosure refers to a specific GUI as illustrated by the several screenshots, it should be understood that other user interfaces (UI) may have the capabilities discussed with reference to the GUI and be employed in the disclosed embodiments to interface between the user and the Model or several models. In addition, because a user may also be a computer system, actions discussed in terms of a GUI or other UI should also be understood as being attributed to computer system interfaces, such as APIs.

### Section 5.1 Features of a Use Case

In some embodiments, the GUI or UI of the use case may include an option to execute a given chemical reaction in an automated or semi-automated laboratory. In one embodiment of this kind, this option enables the user to confirm or test Model predictions. In an embodiment, the Use case may involve an application programming interface (API) to communicate with laboratory hardware. In some embodiments of this kind, the results of the performed experiments are shown in the UI to the user. In some embodiments of this kind, the results of the performed experiments can be added to the Dataset, e.g., at step 160.

In some embodiments the reaction reactants and product together with the reaction conditions predicted by the Model are passed via an API to another software which uses the input to generate a user-readable protocol of the synthesis and/or automated laboratory hardware executable protocol. In some embodiments, such a protocol comprises a sequence of one or more steps, where each step can be performed using a piece of laboratory equipment. In some embodiments, the instruction to execute such a step is provided to the relevant piece of laboratory equipment via an API. Examples of the laboratory hardware that can be instructed by the protocol include automated liquid dispensers, automated solid dispensers, multichannel pipettes, reagent dispensers, robotic arms with grippers moving the plates, or vessels, or racks of vessels., plate sealing devices, vessel capping-decapping devices, gas/vacuum valves, magnetic stirrers, orbital shakers, cooling/heating devices, centrifuges, evaporators, filtering devices, buffers exchanging devices, magnetic modules (for magnetic bead-based chemistry), peristaltic pumps, syringe pumps, vacuum pumps, gas generators, gas compressors, conveyor belts, rail based plate (or vesser, or rack) movers, car-like plate (or vesser, or rack) movers (including partially autonomous devices, e.g. ROVER by Formulatrix). The user may supervise and influence the generated protocol(s) by for example excluding certain reactions from the generated protocol.

In some embodiments, the Model predictions in the Use case are shown only when the confidence of such a prediction is above a certain threshold (according to the Model outputs, see Section 2.5 about the Model), with the end goal of selecting only the most confident ones. This invention is particularly useful in the context of the whole System and its potential applications. The goal of the user might be already satisfied by narrowing down the Model only o a subset of chemical space, but narrowing down to this subspace of only reactions above a threshold confident can further increase the reliability of the Model significantly.

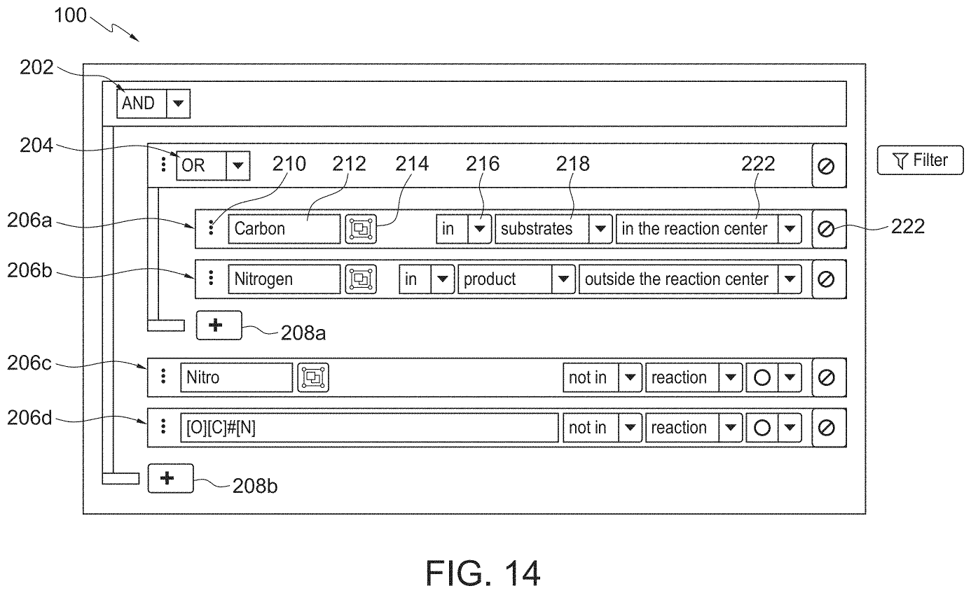

In some embodiments, the graphical interface of Use case can include a GUI enabling showing reactions from the Dataset and potentially executing complex queries against it to find relevant reactions for the user. In one embodiment of this kind, the user can interact via GUI and build queries that reflect what reactions from the Dataset should be fetched for him. In some embodiments, the query may be defined as (potentially) a nested structure of logical constraints such as whether or not a given chemical structure is present in any of the substrates. FIG. 14 shows one such possible embodiment.

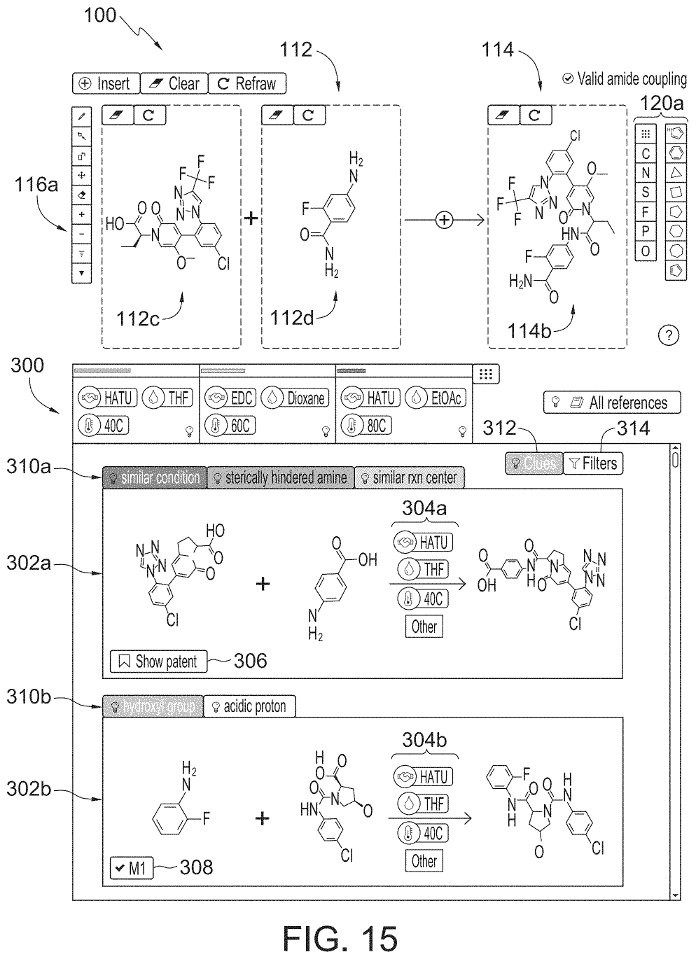

In some embodiments, the Model predictions can be shown together with selected Scientific Arguments (see text below on Scientific Arguments). In one embodiment, the Model predictions are shown together with reactions (referred to as reference reactions) from the Dataset, which can be shown together with a short textual description that explains why a given example is relevant for a given Model prediction. An example of how this embodiment can be implemented in the GUI is shown in FIG. 15.

In some embodiments, Model predictions are shown along with additional outputs designed to increase the interpretability of the Model See Section 2.5 for more details on forms of such explanations. Some examples include showing Scientific Arguments or a list of related reference reactions from the Dataset. In some embodiments of this kind, users may be asked for an opinion on how useful or convincing for them the provided explanation was.

In an embodiment, a user viewing any machine learning model output for a given chemical reaction, in any graphical interface, may be shown user-readable explanations (to which we refer as Scientific Arguments) in the manner that one chemist would explain why a given chemical reaction is plausible or implausible.

In one embodiment of this kind, one or more examples from a dataset (for example the training dataset used to train a given model) are shown to the user if they satisfy a given criterion (defined manually or automatically), along with a text description of such criterion. Examples of such criteria, in the context of machine learning models predicting outcomes of chemical reactions, include: (a) a chemical reaction which has a higher similarity than a defined threshold value; (b) a chemical reaction that has the same user-interpretable chemical feature, such as steric hindrance or electronic density distribution; (c) a chemical reaction that has a similar estimated or measured magnitude of the energy barrier (similar activation energy).

In one embodiment of this kind, a Scientific Argument can be based on a summary- of model performance on any set of compounds (e.g. the Target Set). For example, an expert can be shown a description of the kind: “the model achieves 80% accuracy predicting high yield reactions on heterocycles of the kinds shown in the picture.”

In some embodiments, the GUI includes a display of Scientific Arguments (as defined in the previous paragraph) in ways as discussed in the previous paragraph.

### Section 5.2. Predicting Reaction Outcomes and the Optimal Conditions for a Selected Reaction

In one embodiment, the Model can be used to predict conditions—such as solvent, temperature, or catalyst—that achieve a high yield of a chemical reaction, or satisfy another user-provided constraint, inserted by the user in a graphical user interface or user interface FIG. 16 shows one instantiation of GUI of this Use case.

In one embodiment, the Model can be used to predict the yield of products in the user-specified chemical reaction by inputting the chemical reaction with added information about conditions to the model (see Section 2.5).

In one embodiment, the Model can be used to estimate the probability that the reaction performed under given conditions will result in the yield of the user provided product above a selected threshold by inputting to the model a fully-specified chemical reaction including the yield information (see Section 2.5).

### Section 5.3. Synthesis Planning Using the Model and with the Optional Control of an Autonomous Chemical Laboratory

Regarding designing synthesis pathways, and controlling an autonomous chemical laboratory, in some embodiments, the Model can be used to design synthesis pathways that end in a user-specified target molecule. In one embodiment of this kind, a synthesis planning algorithm (such as Retro* or AiZynthFinder) can be modified (examples discussed in the next paragraph) in a number of ways such that Model outputs influence the final designed synthesis plan.

In one embodiment of this kind, a synthesis planning algorithm may be modified so that the predicted yield and the associated confidence for reactions involved in a synthesis plan impact the prioritization of the synthesis plan with respect to other synthesis plans. In one embodiment of this kind, Retro* or AiZynthFinder synthesis planning algorithms are used. In one embodiment of this kind, the score assigned to reactions by the algorithm (during its operation) includes one or more factors that include the predicted yield for the reactions and confidence about the predictions (see also Section 2.5). The outputs of synthesis planning can be shown in the form of a GUI to the user, or can be read programmatically via an API.

In one such embodiment, the synthesis planning can be used to steer operations of an automated or semi-automated laboratory, suggesting the exact sequence of chemical reactions along with high yielding conditions.

In one embodiment, a GUI allows the end user to send a given chemical reaction for execution in an automated or semi-automated laboratory.

In one embodiment, a separate machine learning model can be used to predict outcomes of a synthesis planning system based on the Model in order to speed up a synthesis planning algorithm. In one embodiment, a neural network based on the Transformer architecture is used to predict the final depth of the synthesis tree predicted by the synthesis planning software or to predict other quantities extracted from the output of the synthesis planning software.

### Section 5.4

In some embodiments, the Model can be used to predict what late stage functionalizations of a given molecule are likely to succeed and under what reaction conditions, and the outputs can be shown in a GUI or accessed using a non-graphical interface. Late stage functionalization is a stage in the drug discovery process, where a promising drug candidate is optimized by making (usually) small modifications to its structure. In embodiment of this kind, the input to the Model is one of the substrates, and the output includes additionally (on top of yield and/or conditions) predicted missing substrate(s) and predicted product(s). In the embodiment, the Model is adapted to output highly likely functionalization chemical reactions along with certainty estimation and condition information by specifying these requirements as constraints as part of the input to the Model (see Section 2.5). In one embodiment, the model predictions can be shown to the user in a GUI as shown in screenshots within this disclosure. In one embodiment, the Model is trained to predict masked out parts of the reaction (e.g. during training input is a reaction with masked substrate and output is the identity of the masked substrate), which allows one to use the Model for late stage functionalization.

### 6. Applications Related to Creating Libraries of Compounds (Physical or Virtual)

In one embodiment, a synthesis plan to synthesize a (large) collection of compounds is designed with the use of the Model to predict reaction conditions of reactions involved in the plan using methods described in Section 5.3. In one embodiment, the synthesis plan is designed following the steps:
a. A user enters a recipe how to synthesize the collection that excludes some pieces of information such as some or all reaction conditions in steps that involve performing a chemical reaction
b. The Model is used by the user to predict the missing pieces of information such as sets of conditions for each step that additionally satisfy user-specified constraints (as described in Section 2.5, we can specify constraints to the Model) such as that the reactions have to be performed at room temperature or that the yield of the desired product must be above a certain threshold.

A DNA encoded library (DEL) is a mixture of a vast (even millions) number of compounds in one solution in which each compound is attached to a tag (usually a strand of DNA) that enables its identification using cheap analytical methods such as DNA sequencing. The envisioned benefit of applying die Model in this context is creating DELs that are more diverse (e.g., include new chemical or chemical reactions for a broader range of molecules) or have higher quality (lower percentage of unexpected/unidentified compounds in the mixture).

A DNA encoded library is usually created by executing a sequence of steps that involve performing chemical reactions. In each step, a mixture of hundreds to millions of lagged compounds is reacted with a single reagent under selected conditions. A key challenge in creating DHLs is that chemical reactions should have a very high yield of the desired product for all the compounds in the mixture and usually have to be performed under conditions compatible with DNA tags, e.g., relatively mild conditions, such as using room temperature to perform the reactions, that do not destroy strands of DNA that are attached to compounds in the mixture.

In one embodiment of the kind above, the procedure is used to design synthesis plans and potentially perform synthesis of a DEL library. In an embodiment of tins kind, users might provide constraints such that the recommended conditions satisfy certain constraints relevant for synthesis of a DEL library such as using low enough temperature to maintain the integrity of DNA tags.

In any embodiment of this kind, the synthesis plan can be executed in any laboratory and the collection of compounds can be physically obtained. In some embodiments of this kind, a human user can modify any parts of the plan, for example by examining model predictions using a user interface that has one or more of the features of any Use Case Application.

In embodiment, the synthesis plan to synthesize a large collection of compounds can be created more automatically using the following steps:
(a) the user specifies a list of “starting” compounds
(b) the user specifies a list of constraints that each reaction in the ultimate synthesis plan should satisfy
(c) the user specifies a maximum number of synthesis steps
(d) all compounds are enumerated such that there is a sequence of reactions generated by the model that ends in this compound as the product;

In another embodiment of this kind, the predicted reactions are shown in a GUI that has one or more of the features of any Use Case Applications.

In one embodiment, a large number of virtual chemical structures can be generated and potentially synthesized by enumerating and applying chemical reactions to commercially available compounds that are predicted to be likely (predicted to achieve a high enough yield with high confidence) according to the Model. In one embodiment of this kind, the Model predictions are filtered down using Model's uncertainty related outputs to only include the most confident predictions.

In one embodiment a GUI or programmatic API is accessible to the user to explore what compounds are part of the collection of compounds.

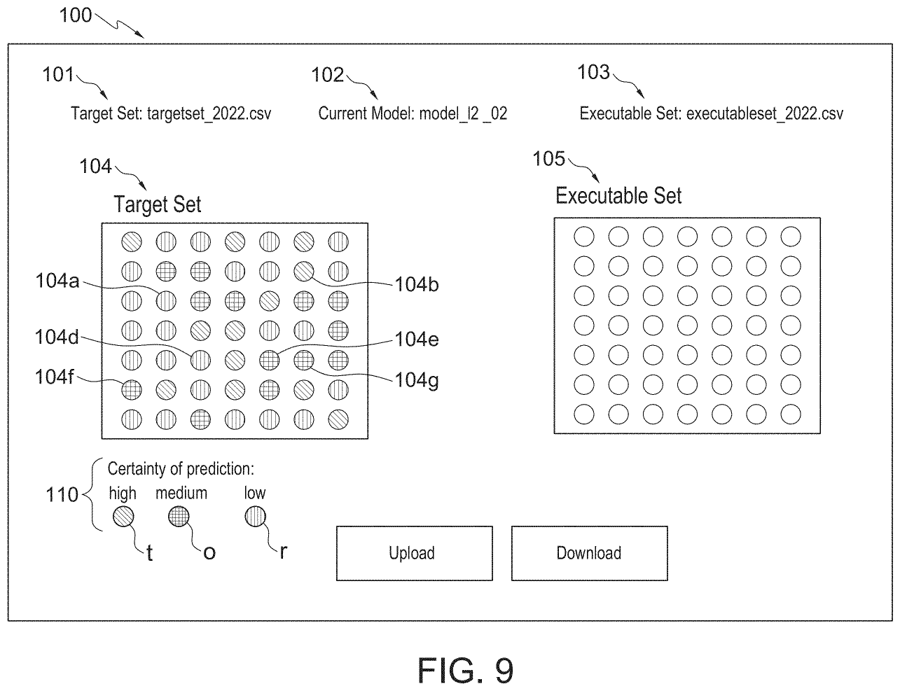

FIG. 9 is a screenshot of part of GUI 100 that can be used to augment user decision making (answering questions, see Section 2.4 for more details) during the Data Collection process. The screenshot of FIG. 9 is a basic view of GUI 100 with indications of a loaded dataset of Target Reactions 101, a Current ML model 102, a loaded data set of reactions 103 that can be executed in the next step “executable set”; a graphical, interactive view 104 of all the reactions, e.g., 104a . . . 104g, from the Target set that may be color-coded with an indication of certainty of the model prediction; a graphical, interactive view 105 of all the reactions from the Executable set (set of chemical reactions that includes reactions possible to perform in the laboratory used in Data Collection), a description 110 of color-coding with, e.g., 104a and 104d coded with an red r “low” certainty, 104b coded with a green t “high” certainty, and with 104g coded with an orange o “medium” certainty); an “upload” button enables the upload of either Target Reactions dataset. Executable reactions dataset, or the model, and a “Download” button will become active after user interaction with the system.

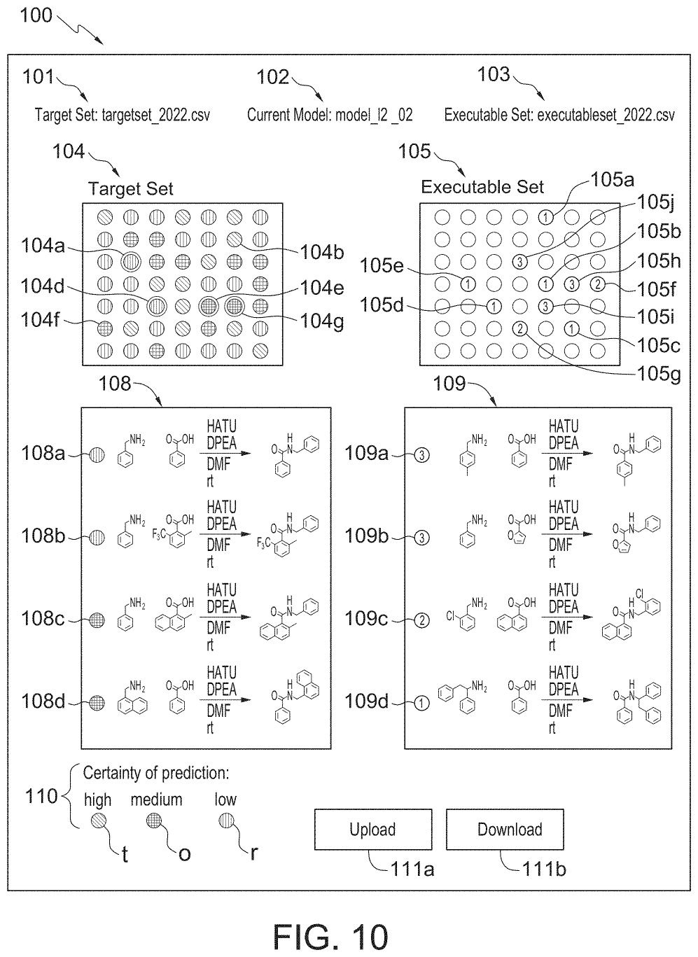

From the screenshot of FIG. 9, as illustrated in the screenshot of FIG. 10, a user can select, one or more reactions 104a . . . 104g from Target set 104. When a reaction is selected, a graphical symbol corresponding to the selected reactions becomes highlighted (circles around 104a, 104d, 104e, and 104g); the selected reactions are displayed as a list 108 (reaction 108a corresponds to 104a, reaction 108b corresponds to 104d, reaction 108c corresponds to 104e, and reaction 108d corresponds to 104g); the ML model identifies reaction(s) 105a . . . 105j from Executable set 105 which would potentially improve the certainty of prediction of selected reactions 104a, 104d, 104e, and 104g, in the scenario that the outcome of indicated reactions from “Executable set” (set of chemical reactions that includes reactions possible to perform in the laboratory used in Data Collection) is known. The number of reactions of the selected subset of Target set 104 that are supported by a given reaction from Executable set is indicated by the numbers 105a . . . 105j, which correspond to the number of reactions front the reactions selected from Target set for which the prediction of ML model would potentially improve if the outcome of this reaction from “Executable set” is known. A list of reactions 109 from Executable set 105 with assigned numbers is presented as reactions 109a . . . 109d (circles 105a . . . 105j correspond to zero or one of reactions 109a . . . 109d). The thus obtained info can be exported to a file of appropriate format via download 111b.

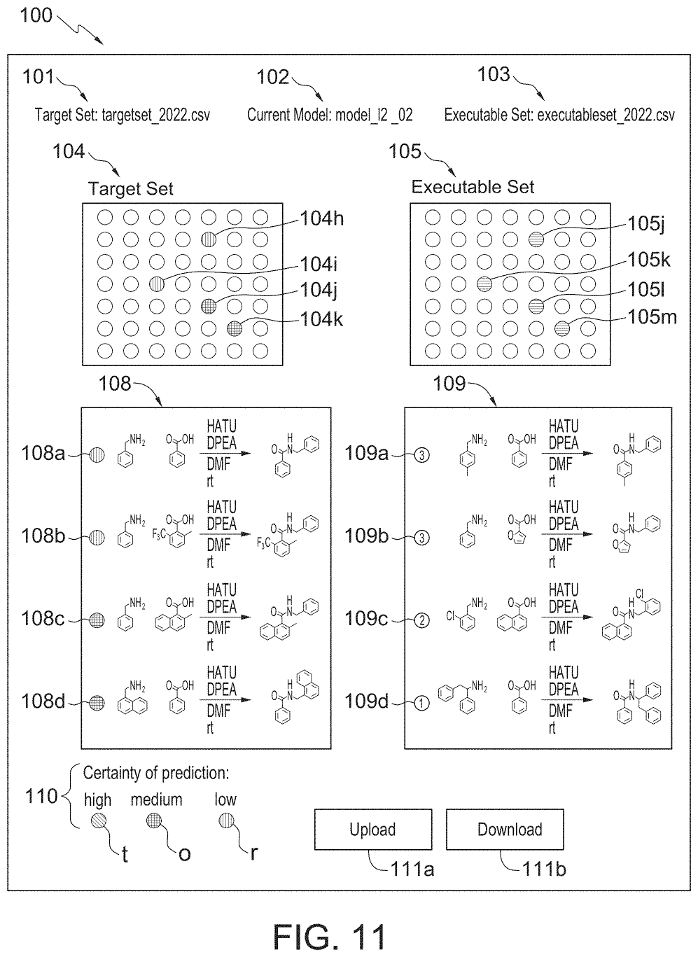

From the screenshot of FIG. 10, as illustrated in the screenshot of FIG. 11, the user can select one or more reactions 105j . . . 105m from Executable set 105, the selected reactions are highlighted as shown, and listed 109 as reactions 109a . . . 109d. The reactions 104h . . . 104k from Target set 104 which can be supported by the selected reactions 104a, 104d, 104e, and 104g are highlighted and listed 108 (reaction 108a corresponds to 104h, reaction 108b corresponds to 104i, reaction 108c corresponds to 104j, and reaction 108d corresponds to 104k). The current certainty of the model on the prediction for highlighted reactions of Target set 104 is displayed by color-codes 110. The thus obtained info can be exported to a file of appropriate format using download 111b.

FIG. 14 is a screenshot of an advanced query builder that can be used by a user in an embodiment involving predicting outcomes and conditions of chemical reactions, the GUI used to augment decision making of a human in Data Collection, and other embodiments. The advanced query builder enables the creation of “parent” filters 202, 204, each of which specifies the logical operator by which the parent filter will be joined with its “children.” Children filter 204, 206c, 206d are children of parent filter 202. Children filter 206a, 206b being children of filter 204. The query builder provides for the creation of new children filters within them with buttons 208a, 208b. A filter can be simultaneously a parent and a child. Filter 204 is a child to filter 202, while being a parent to filters 206a, 206b. All filters apart from the “root” filter 202 and all the filter elements can be freely rearranged via drag and drop functionality 201. Each filter can be specified as either one or more functional groups selected from a predefined set 206a . . . 206c or custom SMARTS 206d. A filter consists of: I. functional group name or custom SMARTS 212. II. a button that, where applicable, opens a separate graphical and/or textual list of functional groups arranged in the subsets of similar chemical nature 214, III. three selection fields that specify the logic (in/not in) 216 and location 218, 220 by which the given filter is applied in the reaction, and IV. a delete button 222.

FIG. 15 is a screenshot 300 of a depiction of reference reactions that can be used by a user in an embodiment of a GUI 100 to an embodiment of a model for predicting outcomes and conditions of chemical reactions, the GUI used to augment decision making of a human in Data Collection, and other embodiments. FIG. 15 illustrates reference reactions 302a, 302b for a single prediction 116a. Each reference reaction 302a, 302b displays its reaction graph with conditions 304a, 304b, a button 306 that toggles the source patent information, or a mark 308 if the reaction was performed in-house, and a list of clues 310a, 310b that explain reasoning behind being selected for this particular prediction. Filtering the reference reactions is possible in two ways: by clues 312 and with custom filters 314 where the filtered results will be an intersection of the two.

FIG. 16 is a screenshot 400 of a reaction editor that can be used by a user in an embodiment of a GUI 100 to an embodiment of a model for predicting outcomes and conditions of chemical reactions, the GUI used to augment decision making of a human in Data Collection, and other embodiments. In FIG. 16, the reaction editor is in the empty state. The editor enables the user to draw a reaction graph consisting of substrates 112, specifically 112e, 112f, product 114, specifically 114c, and reaction conditions 402. Editor buttons 120a allow adding atoms and whole substructures. A popup 404 provides buttons 406a, 406b for starting the reaction graph from a predefined template. Reaction validity (validity here is not something predicted by the model but rather refers to logical validity, for example that carbon is not attached to more than 4 atoms, which would not be physically possible) is checked live while editing and the status 120a is displayed to the user. A predict conditions button 408 to the results view (FIG. 15) that shows sets of predicted conditions for the specified reaction along with other outputs of the Model such as yield. Predict conditions button 408 is disabled until a valid reaction has been specified.

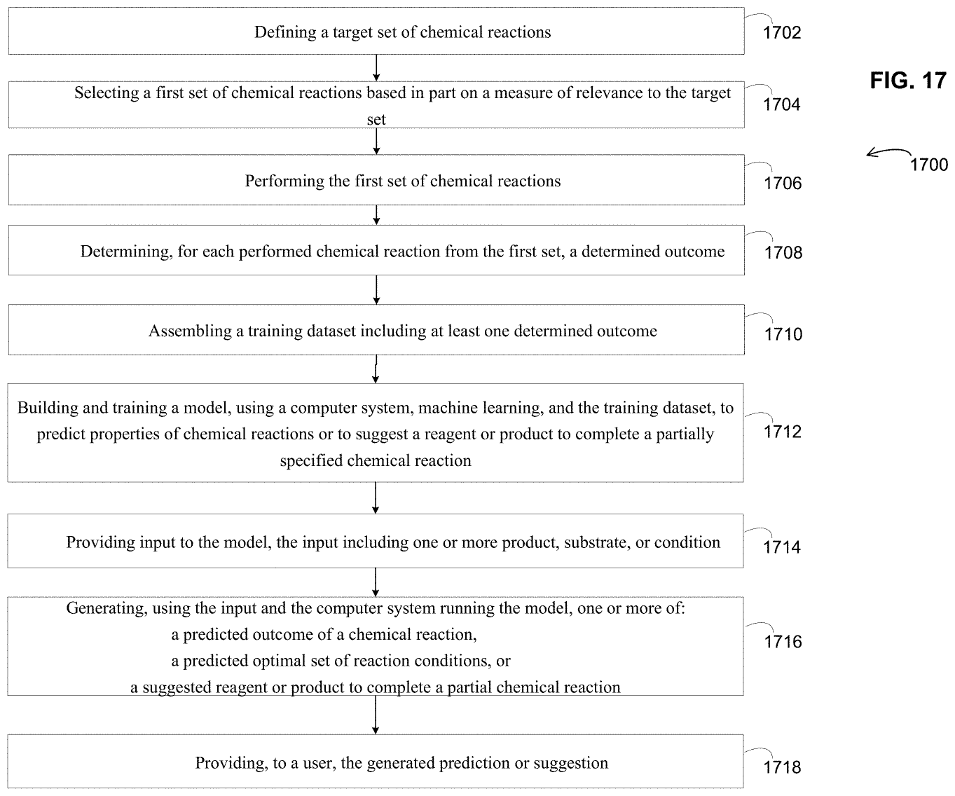

FIG. 17 is a flowchart illustrating an embodiment of a method 1700 for predicting outcomes and conditions of chemical reactions. In method 1700, in a step 1702, a target set of chemical reactions is defined. In a step 1704, a first set of chemical reactions is selected based in part on a measure of relevance to the target set. In a step 1706, the first set of chemical reactions is performed. In a step 1708, an outcome is determined for each performed chemical reaction from the first set. In a step 1710, a training dataset including at least one determined outcome is assembled. In a step 1712, a model is built and trained, using a computer system, machine learning, and the training dataset, to predict properties of chemical reactions or to suggest a reagent or product to complete a partially specified chemical reaction or both. Furthermore, method 1700 may include steps 1714 through 1718. In step 1714 input is provided to the model, the input including one or more product, substrate, or condition. In step 1716, one or more of the following is generated using the input and the computer system running the model: a predicted outcome of a chemical reaction, a predicted optimal set of reaction conditions, or a suggested reagent or product to complete a partial chemical reaction. In step 1718, a user is provided with any generated prediction or suggestion.

Generally, a property of a chemical reaction may be understood to include any characteristic or outcome of a reaction, such as; a reactant, a product, a reaction condition, and a yield.

Generally, in any embodiment, there can be one or more separate GUI implemented, each geared at a different functionality and potentially used by different users, and potentially not communicating with each other. In particular, in embodiments involving use for reaction condition recommendation or steering an automated laboratory, the GUI used for these uses by some users may be separate from the GUI used by the user to steer Data Collection Method.

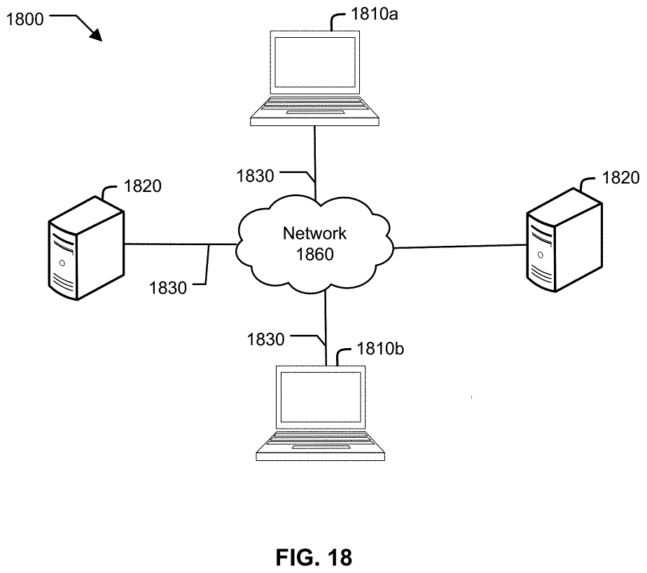

FIG. 18 is an exemplary block diagram depicting an embodiment of system for implement embodiments of methods of the disclosure, e.g., as described with reference to the previous figures. In FIG 18, computer network 1800 includes a number of computing devices 1810a-1810b, and one or more server systems 1820 coupled to a communication network 1860 via a plurality of communication links 1830. Communication network 1860 provides a mechanism for allowing the various components of distributed network 1800 to communicate and exchange information with each other.

Communication network 1860 itself is comprised of one or more interconnected computer systems and communication links. Communication links 1830 may include hardwire links, optical links, satellite or other wireless communications links, wave propagation links, or any other mechanisms for communication of information Various communication protocols may be used to facilitate communication between the various systems shown in FIG. 18. These communication protocols may include TCP/IP, UDP, HTTP protocols, wireless application protocol (WAP), BLUETOOTH, Zigbee, 802.11, 802.15, 6LoWPAN, LiFi, Google Weave, NFC, GSM, CDMA, other cellular data communication protocols, wireless telephony protocols, Internet telephony, IP telephony, digital voice, voice over broadband (VoBB), broadband telephony. Voice over JP ( VoIP), vendor-specific protocols, customized protocols, and others. While in one embodiment, communication network 1860 is the Internet, in other embodiments, communication network 1860 may be any suitable communication network including a local area network (LAN), a wide area network (WAN), a wireless network, a cellular network, a personal area network, an intranet a private network, a near field communications (NFC) network, a public network, a switched network, a peer-to-peer network, and combinations of these, and the like.

In an embodiment, the server 1820 is not located near a user of a computing device, and is communicated with over a network. In a different embodiment, the server 1820 is a device that a user can carry upon his person, or can keep nearby. In an embodiment, the server 1820 has a large battery to power long distance communications networks such as a cell network or Wi-Fi. The server 1820 communicates with the other components of the system via wired links or via low powered short-range wireless communications such as BLUETOOTH. In an embodiment, one of the other components of the system plays the role of the server, e.g., the PC 1810b.

Distributed computer network 1800 in FIG. 18 is merely illustrative of an embodiment incorporating the embodiments and does not limit the scope of the invention as recited in the claims. One of ordinary skill in the an would recognize other variations, modifications, and alternatives. For example, more than one server system 1820 may be connected to communication network 1860. As another example, a number of computing devices 1810a-1810b may be coupled to communication network 1860 via an access provider (not shown) or via some other server system.

Computing devices 1810a-1810b typically request information from a server system that provides the information Server systems by definition typically have more computing and storage capacity than these computing devices, which are often such things as portable devices, mobile communications devices, or other computing devices that play the role of a client in a client-server operation. However, a particular computing device may act as both a client and a server depending on whether the computing device is requesting or providing information. Aspects of the embodiments may be embodied using a client-server environment or a cloud-cloud computing environment.

Server 1820 is responsible for receiving information requests from computing devices 1810a-1810b, for performing processing required to satisfy the requests, and for forwarding the results corresponding to the requests back to the requesting computing device The processing required to satisfy the request may be performed by server system 1820 or may alternatively be delegated to other servers connected to communication network 1860 or to other communications networks. A server 1820 may be located near the computing devices 1810 or may be remote from the computing devices 1810. A server 1820 may be a hub controlling a local enclave of things in an internet of things scenario.

Computing devices 1810a-1810b enable users to access and query information or applications stored by server system 1820. Some example computing devices include portable electronic devices (e.g., mobile communications devices) such as the Apple iPhone®, the Apple iPad®, the Palm Pre™, or any computing device running the Apple iOS™, Android™ OS, Google Chrome OS, Symbian OS®, Windows 10, Windows Mobile® OS, Palm OS® or Palm Web OS™, or any of various operating systems used for Internet of Things (IoT) devices or automotive or other vehicles or Real Time Operating Systems (RTOS), such as the RIOT OS, Windows 10 for IoT, WindRiver VxWorks, Google Brillo, ARM Mbed OS, Embedded Apple iOS and OS X, the Nucleus RTOS, Green Hills Integrity, or Contiki, or any of various Programmable Logic Controller (PLC) or Programmable Automation Controller (PAC) operating systems such as Microware OS-9. VxWorks, QNX Neutrino, FreeRTOS, Micrium μC/OS-II, Micrium μC/OS-III, Windows CE, TI-RTOS, RTEMS. Other operating systems may be used. In a specific embodiment, a “web browser” application executing on a computing device enables users to select, access, retrieve, or query information and/or applications stored by server system 1820. Examples of web browsers include the Android browser provided by Google, the Safari® browser provided by Apple, the Opera Web browser provided by Opera Software, the BlackBerry® browser provided by Research In Motion, the Internet Explorer® and Internet Explorer Mobile browsers provided by Microsoft Corporation, the Firefox® and Firefox for Mobile browsers provided by Mozilla®, and others.

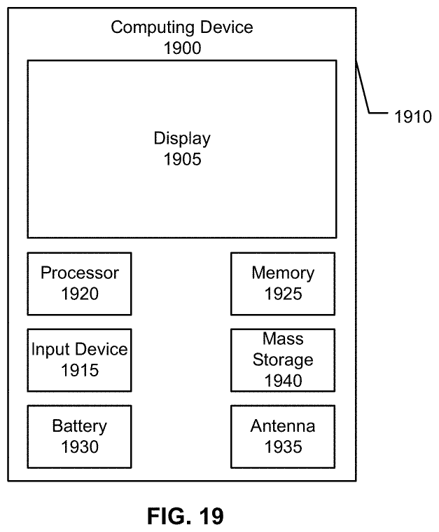

FIG. 19 is an exemplary block diagram depicting a computing device 1900 of an embodiment. Computing device 1900 may be any of the computing devices 1810a, 1810b, 1820 from FIG. 18. Computing device 1900 may include a display, screen, or monitor 1905, housing 1910, and input device 1915. Housing 1910 houses familiar computer components, some of which are not shown, such as a processor 1920, memory 1925, battery 1930, speaker, transceiver, antenna 1935, microphone, ports, jacks, connectors, camera, input/output (I/O) controller, display adapter, network interface, mass storage devices 1940, various sensors, and the like.

Input device 1915 may also include a touchscreen (e.g., resistive, surface acoustic wave, capacitive sensing, infrared, optical imaging, dispersive signal, or acoustic pulse recognition), keyboard (e.g., electronic keyboard or physical keyboard), buttons, switches, stylus, or combinations of these.

Mass storage devices 1940 may include flash and other nonvolatile solid-state storage or solid-state drive (SSD), such as a flash drive, flash memory, or USB flash drive. Other examples of mass storage include mass disk drives, floppy disks, magnetic disks, optical disks, magneto-optical disks, fixed disks, hard disks. SD cards, CD-ROMs, recordable CDs, DVDs, recordable DVDs (e.g., DVD-R, DVD+R, DVD-RW, DVD+RW, HD-DVD, or Blu-ray Disc), battery-backed-up volatile memory, tape storage, reader, and other similar media, and combinations of these.

Embodiments may also be used with computer systems having different configurations, e.g., with additional or fewer subsystems. For example, a computer system could include more than one processor (i.e., a multiprocessor system, which may permit parallel processing of information) or a system may include a cache memory. The computer system shown in FIG. 19 is but an example of a computer system suitable for use with the embodiments. Other configurations of subsystems suitable for use with the embodiments will be readily apparent to one of ordinary skill in the art. For example, in a specific implementation, the computing device is a mobile communications device such as a smartphone or tablet computer. Some specific examples of smartphones include the Droid Incredible and Google Nexus One, provided by HTC Corporation, the iPhone or iPad, both provided by Apple, and many others. The computing device may be a laptop or a netbook. In another specific implementation, the computing device is a non-portable computing device such as a desktop computer or workstation.

A computer-implemented or computer-executable version of the program instructions useful to practice the embodiments may be embodied using, stored on, or associated with computer-readable medium. A computer-readable medium may include any medium that participates in providing instructions to one or more processors for execution, such as memory 1925 or mass storage 1940 Such a medium may take many forms including, but not limited to, nonvolatile, volatile, transmission, non-printed, and printed media. Nonvolatile media includes, for example, flash memory, or optical or magnetic disks. Volatile media includes static or dynamic memory, such as cache memory or RAM. Transmission media includes coaxial cables, copper wire, fiber optic lines, and wires arranged in a bus. Transmission media can also take the form of electromagnetic, radio frequency, acoustic, or light waves, such as those generated during radio wave and infrared data communications.

For example, a binary, machine-executable version, of the software useful to practice the embodiments may be stored or reside in RAM or cache memory, or on mass storage device 1940. The source code of this software may also be stored or reside on mass storage device 1940 (e.g., flash drive, hard disk, magnetic disk, tape, or CD-ROM). As a further example, code useful for practicing the embodiments may be transmitted via wires, radio waves, or through a network such as the Internet. In another specific embodiment, a computer program product including a variety of software program code to implement features of the embodiment is provided.

Computer software products may be written in any of various suitable programming languages, such as C, C++, C#, Pascal, Fortran, Perl, Matlab (from MathWorks, www.mathworks.com), SAS, SPSS, JavaScript, CoffeeScript, Objective-C, Swift, Objective-J, Ruby, Rust, Python, Erlang, Lisp, Scala, Clojure, and Java. The computer software product may be an independent application with data input and data display modules. Alternatively, the computer software products may be classes that may be instantiated as distributed objects. The computer software products may also be component software such as Java Beans (from Oracle) or Enterprise Java Beans (EJB from Oracle).

An operating system for the system may be the Android operating system, iPhone OS (i.e., iOS), Symbian, BlackBerry OS, Palm web OS, Bada, MeeGo, Maemo, Limo, or Brew OS. Other examples of operating systems include one of the Microsoft Windows family of operating systems (e.g., Windows 95, 98, Me, Windows NT, Windows 2000, Windows XP, Windows XP x64 Edition, Windows Vista, Windows 10 or other Windows versions, Windows CE, Windows Mobile, Windows Phone, Windows 10 Mobile), Linux, HP-UX, UNIX, Sun OS, Solaris, Mac OS X, Alpha OS, AIX, IRIX32, or IRIX64, or any of various operating systems used for Internet of Things (IoT) devices or automotive or other vehicles or Real Time Operating Systems (RTOS), such as the RIOT OS, Windows 10 for IoT, WindRiver VxWorks, Google Brillo, ARM Mbed OS, Embedded Apple iOS and OS X, the Nucleus RTOS, Green Hills integrity, or Contiki, or any of various Programmable Logic Controller (PLC) or Programmable Automation Controller (PAC) operating systems such as Microware OS-9, VxWorks, QNX Neutrino, FreeRTOS, Micrium μC/OS-II, Micrium μC/OS-III, Windows CE, TI-RTOS, RTEMS. Other operating systems may be used.

Furthermore, the computer may be connected to a network and may interface to other computers using this network. The network may be an intranet, internet, or the Internet, among others. The network may be a wired network (e.g., using copper), telephone network, packet network, an optical network (e.g., using optical fiber), or a wireless network, or any combination of these. For example, data and other information may be passed between the computer and components for steps) of a system useful in practicing the embodiments using a wireless network employing a protocol such as Wi-Fi (IEEE standards 802.11, 802.11a, 802.11b, 802.11e, 802.11g, 802.11i, and 802 11n, just to name a few examples), or other protocols, such as BLUETOOTH or NFC or 802.15 or cellular, or communication protocols may include TCP/IP, UDP, HTTP protocols, wireless application protocol (WAP), BLUETOOTH, Zigbee, 802.11, 802.15, 6LoWPAN, LiFi, Google Weave, NFC, GSM, CDMA, other cellular data communication protocols, wireless telephony protocols or the like. For example, signals from a computer may be transferred, at least in part, wirelessly to components or other computers.

The following paragraphs set forth enumerated embodiments.

Embodiment 1. A method comprising:

defining a target set of chemical reactions;

selecting a first set of chemical reactions based in part on a measure of relevance to the target set;

performing the first set of chemical reactions;

determining, for each performed chemical reaction from the first set, an outcome;

assembling a training dataset including at least one determined outcome;

building and training a model, using a computer system, machine learning, and the training dataset, that predicts properties or outcomes of chemical reactions, or that suggests one or more reactant, reaction condition, or product to complete an incomplete chemical reaction.

Embodiment 2. The method of embodiment 1, further comprising.

providing input to the model, the input including one or condition;

generating, using the input and the computer system running the model, one or more of:
a predicted outcome of a chemical reaction,
a predicted set of reaction conditions, or
a suggested one or more of each of reactant, reaction condition, or product to complete an incomplete chemical reaction, or
a predicted outcome for the incomplete chemical reaction; and

providing, to a user, the generated prediction or suggestion

Embodiment 3. The method of embodiment 2, wherein the providing steps are performed using a user interface.
Embodiment 4. The method of embodiment 1, further comprising:

after the step of building and training the model, determining to repeat one or more of the steps of. selecting a first set of chemical reactions, performing the first set of chemical reactions, determining a determined outcome, or assembling a training dataset; and

repeating the one or more steps.

Embodiment 5. The method of embodiment 4, wherein the determining to repeat one or more of the steps is performed automatically by the computer system.
Embodiment 6. The method of embodiment 1, wherein:

the first set of chemical reactions is performed using automated or semi-automated laboratory equipment, and

the determining a determined outcome includes performing measurements of each post-reaction mixture and quantification using software processing to determine at least one yield.

Embodiment 7. The method of embodiment 1, wherein:

defining the target set includes defining the target set by specifying one or more constraints that chemical reactions of the target set must satisfy.

Embodiment 8. The method of embodiment 1, wherein defining the target set includes:

providing, by a user: a list of chemical compounds, or one or more constraints on chemical compounds, or one or more constraints on reactions; and

defining the target set as hypothetical reactions that satisfy the constraints that have a product from the list of chemical compounds or a product that satisfies the constraints.

Embodiment 9. The method of embodiment 1, wherein the first set of chemical reactions is selected based in part on one or more factors including:

(a) a chemical similarity of reactions in the set to reactions in the target set,

(b) a chemical similarity between reactions in the set.

(c) a price of reagents or reactants in the first chemical reaction;

(d) an availability of reagents or reactants in the first chemical reaction;

(e) one or more predictions of the model when inputted the chemical reactions; or

(f) one or more estimations of uncertainty about predictions of the model when inputted the chemical reactions.

Embodiment 10. The method of embodiment 4, further comprising:

providing input to the model, the input including one or more product, substrate, or condition from either:
the target set,
a set of chemical reactions more chemically complex than the first set of reactions, or
a part of the performed reactions that were not used to train the model;

generating, using the input and the computer system running the model, one or more of:
a predicted outcome of a chemical reaction,
a predicted optimal set of reaction conditions, or
a suggested reagent or product to complete a partial chemical reaction; comparing the generated prediction or suggestion to a reaction from the target set; arid determining a level of performance of the model based on the comparison, wherein: the determining to repeat one or more of the steps is based on the level of performance.
   Embodiment 11. The method of embodiment 1, wherein the training dataset includes one or more of:

(i) an outcome of a chemical reaction determined from performing the chemical reaction;

(ii) an outcome of a chemical reaction extracted by the computer system from text,

(iii) an outcome of a computer program (hat simulates outcomes of chemical reactions using molecular modeling; or

(iv) an outcome of a chemical reaction recorded in an electronic lab notebook

Embodiment 12. The method of embodiment 2, wherein generating, using the input and the computer system running the model, one or more of:
a predicted outcome of a chemical reaction,
a predicted optimal set of reaction conditions, or
a suggested reagent or product to complete a partial chemical reaction; includes:

generating, using the input and the computer system running the model, a plurality of predicted outcomes for a chemical reaction or a plurality of sets of optimal conditions for performing the chemical reaction;

filtering, by the model, the plurality of predicted outcomes or the plurality of sets of optimal conditions to eliminate predicted outcomes with a level of certainty below a threshold level of certainty or to eliminate sets of optimal conditions with a level of performance below a threshold level of performance.

Embodiment 13. The method of embodiment 1, wherein when a human is asked a question that influences the method in any way, be is shown a user interface comprising of one or more of the following features:

(a) shown performance of the model according to any metric;

(b) predictions of the model are supplemented by examples fetched from the dataset used to train the model;

(c) any feature that can be also present in the user interface used to interact with the model by the user;

Embodiment 14. The method of embodiment 13, whereas the set of reactions is selected based also on a factor that includes a numerical score assigned by a human who answers a question regarding one or more chemical reactions using the user interface.
Embodiment 15. The method of embodiment 1, wherein synthesis of a new collection of compounds is planned and potentially performed by:

user inputting a partially specified recipe for how to synthesis the collection of compounds that is not yet ready for performing;

generating using the model missing information for the recipe that satisfies user provided constraints;

optionally, displaying the recipe and/or the collection of compounds in a user interlace

optionally, performing the recipe to synthesize the collection of compounds

Embodiment 16. The method of embodiment 15, wherein the collection of compounds is such that the collection of compounds is dissolved in a single solution and each compound is identified by a strand of DNA or another set. of atoms enabling its identification.
Embodiment 17. The method of embodiment 15, wherein the user provided constraints include one or more of:

(a) yield of reaction is above a given threshold

(b) conditions satisfy certain logical constraints.

Embodiment 18. The method of embodiment 15, wherein the synthesis plan is generated using the following steps:

(a) the user specifies a list of starting compounds;

(a) the user specifies a list of constraints that each reaction in the ultimate synthesis plan should satisfy;

(b) the user specifies a maximum number of synthesis steps, and

(c) all compounds are enumerated such that there is a sequence of reactions generated by the model that ends in this compound as the product.

Embodiment 19. The method of embodiment 1, further comprising:

inputting, by a user, a chemical reaction that is partially specified (for example has only specified product and one of the substrates),

completing the reaction, using the model after the model is additionally trained to predict missing parts of the reaction; and

generating, by the model, predictions about the optimal conditions and yields for the completed reaction.

Embodiment 20. The method of embodiment 1, further comprising:

inputting by the user a target molecule structure;

generating, using the model and a synthesis planning algorithm that utilizes the model and any synthesis planning software; predictions, one or more synthesis pathways for the target molecule structure; and

displaying, using a user interface, the predicted synthesis pathway.

Embodiment 21. The method of embodiment 20, further comprising:

generating synthesis pathways using a retrosynthesis algorithm that uses the predicted optimal conditions by the model as a factor influencing the choice of the synthesis pathway.

Embodiment 22. The method of embodiment 1, whereas any of the following holds:

(i) performing chemical reactions is done using automated solid and/or liquid dispensers;

(ii) performing chemical reactions is done in multi well plates of standardized dimensions, with separate reactions in each well;

(iii) performing chemical reactions is done in vessels made of glass, plastic, or metal, each reaction in a separate vessel and vessels are organized spatially in a rack;

(iv) performing chemical reactions is done with stirring of the reaction mixtures with magnetic stirring bars or by orbital shaker; or

(v) performing chemical reactions is done with heating or cooling of the reaction mixture in a heating/cooling block or inside a thermoshaker.

Embodiment 23. The method of embodiment 1, whereas analysis of the amount of expected product in the post-reaction mixture is achieved by any combination of:

(i): liquid chromatography combined with one or more detectors listed below:

single or multi-wavelength UV-Vis detector,

fluorescence detector,

evaporative light scattering detector (ELSD),

charged aerosol detector (CAD),

radiometric detector,

electrochemical detector,

chemiluminescent nitrogen detector, or

mass spectrometer;

(ii) liquid chromatography with manual or automated fraction collection and subsequent determination of the quantity of the product in the combined fractions containing the expected product by any applicable analytical method;

(iii) method as in (ii) but with isolation of the product by solid phase extraction (SPE) technique;

(iv) method as in (i) but in the MISER (multiple injections in a single experimental run) mode;

(v) MALDI-MS or Echo-MS analysis of the appropriately prepared analytical samples of post-reaction mixtures—without separation of components; or

(vi) nuclear magnetic resonance (NMR) spectroscopy of unprocessed or anyhow processed post-reaction mixtures.

Embodiment 24. The method of embodiment 1, whereas any of the following holds:
(i) the signal (data) from the analytical instrument acquired for the analytical sample of the post-reaction mixture is processed by a dedicated computer program in order to automatically quantify the expected product;
(ii) any computational method or ML model is used to predict the level of analytical signal for expected reaction products;
(iii)any computational method or ML model uses the analytical signals of internal analytical standard(s) and reaction product to quantify the amount of product in the analytical sample.
Embodiment 25. The method of embodiment 1, wherein the machine learning model has any of the following features:

(a) The model architecture is a sequence to sequence deep neural network such as the Transformer architecture;

(b) The model is based on ensembling;

(c) The model output includes additionally measures of uncertainty of other outputs;

(d) Model uncertainty is computed based on individual outputs of ensemble members, or

(e) The model accepts as input a set of logical constraints to be satisfied by the output, and produces output that satisfies these constraints.

Embodiment 26. The method of embodiment 1, further comprising:

a user interface that enables the user to explore and view reactions that are part of the dataset used to train the model.

Embodiment 27. The method of embodiment 1, further comprising:

a user interface enabling the user to execute a selected reaction in an external semi or fully automated laboratory.

Embodiment 28. The method of embodiment 27, whereas exploration is enabled by a mechanism enabling executing queries against the database that surface reactions that satisfy user provided constraints such as the chemical structure present in the reaction.
Embodiment 29. The method of embodiment 1, further comprising;
a user interface enabling the user to programmatically use the software by, for example, encoding in a computer medium a set of instructions that instructs the software to perform any actions that the user could have executed manually.
Embodiment 30. The method of embodiment 2, wherein
the model is trained to provide predictions or suggestions that satisfy any of the following constraints:

(a) a level of confidence of predictions is above certain threshold

(b) logical constraints to be satisfied by predicted conditions or outcomes

(c) logical constraints to be satisfied by suggested reactions

providing input to the model, the input including one or more product, substrate, or condition, and includes additionally any of the following

(a) a selected level of confidence threshold

(b) a selected logical constraint to be satisfied by predicted conditions or outcomes

(c) a selected logical constraint to be satisfied by suggested reactions

generating, using the input and the computer system running the model, one or more of.
a predicted outcome of a chemical reaction with potentially associated level of confidence matching the input level of confidence,
a predicted optimal set of reaction conditions with potentially associated level of confidence matching the input level of confidence, or
a suggested reactant, reaction condition, or product to complete a partial chemical reaction with potentially associated level of confidence matching the input level of confidence, and
providing, to a user, the generated prediction or suggestion.
Embodiment 31. The method of embodiment 1, further comprising:

inputting by the user a target molecule structure;

generating, using the model and a synthesis planning algorithm that utilizes the model and synthesis planning software;

predictions of one or more synthesis pathways for the target molecule structure; and

optionally, displaying, using a user interface, the predicted synthesis pathway, or

optionally, executing the synthesis plan using automated or semi-automated laboratory.

Embodiment 32. The method of embodiment 1, whereas any of the following holds:
(i) the signal (data ) from the analytical instrument acquired for the analytical sample of the post-reaction mixture is processed by a dedicated computer program in order to automatically quantify the expected product.
(ii) any computational method or ML model is used to predict the level of analytical signal for expected reaction products, or
(iii) any computational method or ML model uses the analytical signals of internal analytical standard(s) and reaction product to quantify the amount of product, in the analytical sample.
Embodiment 33. The method of claim 1, further comprising planning a synthesis of a compound or a collection of compounds by:

designing by the user or the first computer system or a second computer system a partially specified recipe for how to synthesize the compound or the collection of compounds; and

generating, using the model, missing information for the recipe that satisfies user provided constraints.

A system comprising at least one processor and memory with instructions that when executed by the at least, one processor cause the system to perform actions according to any of embodiments 1-33 above.

A non-transitory, computer-readable medium comprising instructions that when executed by a processor of a computing device cause the computing device to perform actions according to any of embodiments 1-33 above.

While the embodiments have been described with regards to particular embodiments, it is recognized that additional variations may be devised without departing from the inventive concept.

The terminology used herein is for the purpose of describing particular embodiments only and is not intended to be limiting of the claimed subject matter. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will further be understood that the terms “comprises” and/or “comprising,” when used in this specification, specify the presence of states features, steps, operations, elements, and/or components, but do not preclude the present or addition of one or more other features, steps, operations, elements, components, and/or groups thereof.

Unless otherwise defined, all terms (including technical and scientific terms) used herein have the same meaning as commonly understood by one having ordinary skill in the art to which the embodiments belong. It will further be understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the embodiments, it will be understood that a number of elements, techniques, and steps are disclosed. Each of these has individual benefit and each can also be used in conjunction with one or more, or in some cases all, of the other disclosed elements, or techniques. The specification and claims should be read with the understanding that such combinations are entirely within the scope of the embodiments and the claimed subject matter.

In the description above and throughout, numerous specific details are set forth in order to provide a thorough understanding of an embodiment of this disclosure. It will be evident, however, to one of ordinary skill in the art, that an embodiment may be practiced without these specific details. In other instances, well-known structures and devices are shown in block diagram form to facilitate explanation. The description of the preferred embodiments is not intended to limit the scope of the claims appended hereto. Further, in the methods disclosed herein, various steps are disclosed illustrating some of the functions of an embodiment. These steps are merely examples and are not meant to be limiting in any way. Other steps and functions may be contemplated without departing from this disclosure or the scope of an embodiment.

1 . A method comprising:
defining a target set of chemical reactions;
selecting a first set of chemical reactions based in part on a measure of relevance to the target set;
performing the first set of chemical reactions;
determining, for each performed chemical reaction from the first set, an outcome;
assembling a training dataset including at least, one determined outcome;
building and training a model, using a first computer system, machine learning, and the training dataset, that predicts properties or outcomes of chemical reactions, or that suggests one or more reactant, reaction condition, or product to complete an incomplete chemical reaction.

2. The method of claim 1, further comprising:
providing input to the model, the input including one or more product, reactant, or reaction condition;
generating, using the input and the computer system running the model, one or more of:
a predicted property or outcome of a chemical reaction,
a predicted set of reaction conditions, or
a suggested one or more of: a reactant a reaction condition, or a product to complete an incomplete chemical reaction; or
a predicted outcome for the incomplete chemical reaction; and
providing, to a user, the generated prediction or suggestion.

3. The method of claim 1, further comprising:
after the step of building and training the model, determining to repeat one or more of the steps of. selecting a first set of chemical reactions, performing the first set of chemical reactions, determining a determined outcome, or assembling a training dataset; and
repeating the one or more steps.

4. The method of claim 3, wherein the determining to repeat one or more of the steps is performed automatically by the first computer system or a second computer system.

5. The method of claim 1, wherein:
the first set of chemical reactions is performed using automated or semi-automated laboratory equipment; and
the determining a determined outcome includes performing measurements of each post-reaction mixture and quantification using software processing to determine at least one yield.

6. The method of claim 1, wherein:
defining the target set includes defining the target set by specifying one or more constraints that chemical reactions of the target, set must satisfy.

7. The method of claim 1, wherein defining the target set includes:
providing, by a user a list of chemical compounds, or one or more constraints on chemical compounds, or one or more constraints on reactions; and
defining the target set as hypothetical reactions that satisfy the constraints that have a product from the list of chemical compounds or a product that satisfies the constraints.

8. The method of claim 1, wherein the first set of chemical reactions is selected based in part on one or more factors including:
(a) a chemical similarity of reactions in the set to reactions in the target set;
(b) a chemical similarity between reactions in the set;
(c) a price of reagents or reactants in the first chemical reaction;
(d) an availability of reagents or reactants in the first chemical reaction;
(e) one or more predictions of the model when inputted the chemical reactions; or
(f) one or more estimations of uncertainty about predictions of the model when inputted the chemical reactions.

9. The method of claim 3, further comprising:
providing input to the model, the input including one or more product, substrate, or condition from either:
the target set,
a set of chemical reactions more chemically complex than the first set of reactions; or
a part of the performed reactions that were not used to train the model;
generating, using the input and the computer system running the model, one or more of:
a predicted outcome of a chemical reaction,
a predicted optimal set of reaction conditions, or
a suggested reagent or product to complete a partial chemical reaction; comparing the generated prediction or suggestion to a reaction from the target set, and determining a level of performance of the model based on the comparison, wherein, the determining to repeat one or more of the steps is based on the level of performance.

10. The method of claim 1, wherein the training dataset includes one or more of:
(i) an outcome of a chemical reaction determined from performing the chemical reaction;
(ii) an outcome of a chemical reaction extracted by a computer system from text;
(iii) an outcome of a computer program that simulates outcomes of chemical reactions using molecular modeling; or
(iv) an outcome of a chemical reaction recorded in an electronic lab notebook.

11. The method of claim 2, wherein generating, using the input and the computer system running the model, one or more of:
a predicted outcome of a chemical reaction,
a predicted optimal set of reaction conditions, or
a suggested reagent or product to complete a partial chemical reaction;
includes:
generating, using the input and the computer system running the model, a plurality of predicted outcomes for a chemical reaction or a plurality of sets of optimal conditions for performing the chemical reaction,
filtering, by the model, the plurality of predicted outcomes or the plurality of sets of optimal conditions to eliminate predicted outcomes with a level of certainty below a threshold level of certainty or to eliminate sets of optimal conditions with a level of performance below a threshold level of performance.

12. The method of claim 1, wherein when a user is asked a question that influences the method in any way, be is shown a user interface comprising of one or more of the following features:
(a) performance of the model according to any metric;
(b) predictions of the model supplemented by examples fetched from the dataset used to train the model; or
(c) any feature that can be also present in the user interface used to interact with the model by the user.

13. The method of claim 12, wherein the set of reactions is selected based also on a factor that includes a numerical score assigned by a human who answers a question regarding one or more chemical reactions using the user interface.

14. The method of claim 1, further comprising planning a synthesis of a compound or a collection of compounds by:
designing by the user or the first computer system or a second computer system a partially specified recipe for how to synthesize the compound or the collection of compounds; and
generating, using the model: missing information for the recipe that satisfies user provided constraints.

15. A system comprising at least one processor and memory with instructions that when executed by the at least one processor cause the system to perform actions including:
receiving a target set of chemical reactions,
receiving a first set of chemical reactions selected based in part on a measure of relevance to the target set;
determining, for each performed chemical reaction from the first set, a determined outcome,
receiving an assembled training dataset including at least one outcome from each chemical reaction from the first set, each at least one outcome determined from a performance of a different chemical reaction from the first set, and
building and training a model, using machine learning, and the training dataset, to predict properties of chemical reactions or to suggest a reagent or product to complete a partially specified chemical reaction.

16. The system of claim 15, the actions further comprising:
receiving input to the model, the input including one or more product, substrate, or condition;
generating, using the input and running the model, one or more of:
a predicted outcome of a chemical reaction,
a predicted optimal set of reaction conditions, or
a suggested reagent or product to complete a partial chemical reaction; and providing, to a user, the generated prediction or suggestion.

17. The system of claim 16, wherein generating, using the input and the computer system running the model, one or more of:
a predicted outcome of a chemical reaction,
a predicted optimal set of reaction conditions, or
a suggested reagent or product to complete a partial chemical reaction;
includes:
generating, using the input and running the model, a plurality of predicted outcomes tor a chemical reaction or a plurality of sets of optimal conditions for performing the chemical reaction, and
filtering, using the model, the plurality of predicted outcomes or the plurality of sets of optimal conditions to eliminate predicted outcomes with a level of certainty below a threshold level of certainty or to eliminate sets of optimal conditions with a level of performance below a threshold level of performance.

18. A non-transitory, computer-readable medium comprising instructions that when executed by a processor of a computing device cause the computing device to perform actions including:
receiving a target set of chemical reactions;
receiving a first set of chemical reactions selected based in pan on a measure of relevance to the target set;
determining, for each performed chemical reaction from the first set, a determined outcome;
receiving an assembled training dataset including at least one outcome from each chemical reaction from the first set, each at least one outcome determined from a performance of a different chemical reaction from the first set; and
building and training a model, using machine learning, and the training dataset, to predict properties of chemical reactions or to suggest a reagent or product to complete a partially specified chemical reaction.

19. The non-transitory computer-readable medium of claim 18, the actions further comprising:
receiving input to the model, the input including one or more product, substrate, or condition,
generating, using the input and running the model, one or more of:
a predicted outcome of a chemical reaction;
a predicted optimal set of reaction conditions, or
a suggested reagent or product to complete a partial chemical reaction; and providing, to a user, the generated prediction or suggestion.

20. The non-transitory computer-readable medium of claim 19, wherein generating, using the input and the computer system running the model, one or more of:
a predicted outcome of a chemical reaction;
a predicted optimal set of reaction conditions, or
a suggested reagent or product to complete a partial chemical reaction; includes:
generating, using the input, and running the model, a plurality of predicted outcomes for a chemical reaction or a plurality of sets of optimal conditions for performing the chemical reaction, and
filtering, using the model the plurality of predicted outcomes or the plurality of sets of optimal conditions to eliminate predicted outcomes with a level of certainty below a threshold level of certainty or to eliminate sets of optimal conditions with a level of performance below a threshold level of performance.
